(19) United States
(12) Patent Application Publication
(10) Pub. No.: US 2026/0194530 A1
(43) Pub. Date: Jul. 9, 2026

(54) MEANS AND METHODS TO DETERMINE CELLULAR AVIDITY

(71) Applicant: LUMICKS CA HOLDING B.V., Amsterdam (NL)

(72) Inventors: Serena Alba DAVOLI, Amsterdam (NL); Mattijs DE GROOT, Amsterdam (NL); Marinus Theodorus Johannes VAN LOENHOUT, Amsterdam (NL); Trillian Ashley GREGG, Amsterdam (NL)

(21) Appl. No.: 18/863,913

(22) PCT Filed: May 12, 2023
(86) PCT No.: PCT/EP2023/062718

§ 371 Date: Nov. 7, 2024

(30) Foreign Application Priority Data
May 13, 2022 (EP) 22173290.2

(51) Int. Cl. G01N 33/68; G01N 3/24; G01N 33/50; G01N 33/569

(52) U.S. Cl. CPC G01N 33/68; G01N 3/24; G01N 33/5005; G01N 33/56972; G01N 2203/0025; G01N 2333/7051; G01N 2333/976

(57) ABSTRACT

The current invention relates to improved methods utilizing cellular avidity, said methods involving applying a force such that cell-cell bonds can be broken, and furthermore detecting the presence or absence of a marker associated with synapse formation in cells that remain bound thereafter. These methods are highly useful in determining cellular avidity and cellular avidity measurements.

### INTRODUCTION

In the art, binding studies have been conducted with cells (e.g. target cells) being attached to a surface, e,g. a glass surface, wherein subsequent further cells (e.g. effector cells) are added to interact (e.g. study the binding thereof) with the attached cells. In particular, such studies were performed to determine or assess cellular avidity, e.g. of effector cells such as CAR-T cells and cancer cells. Cellular avidity is then subsequently determined by exerting a force on the cells that interacted with the attached cells and by subsequently analysing cells that detach and/or remain attached to the cells attached to the surface (see e.g. FIG. 2).

However, a problem that can be observed with cells attached to a surface, is that in addition to specific binding of the cells to the attached cells, background binding, not necessarily attributable to specific binding, may play a role in the binding of cells to the cells attached to the surface. Such background binding, to which also can be referred to as aspecific binding, can be observed for example when control cells are tested under the same conditions of binding studies conducted with the cells of interest and when after the force has been exerted a substantial binding is shown between cells attached to the surface and subsequent further cells. When such binding is relatively high as compared with specific binding, e.g. when studying the interaction between cells that are to specifically interact (e.g. an effector cell with a CAR targeting an antigen expressed by a cancer cell), it becomes much more difficult to differentiate between aspecific and specific interactions and to detect variation in cellular avidity, e.g. when comparing different receptors, e.g. CARs and/or different target cells, e.g. different cancer cells expressing the same antigen the CAR is targeting.

Hence, there is a need in the art to provide for improved means and methods for utilizing and determining cellular avidity, in particular when substantial background binding may occur as well. Moreover, there is also a need in the art to obtain cells that have remained bound, and attached to a surface, after a cellular avidity measurement, as it may be of interest to further study these cells and or further utilize these cells in subsequent means and methods.

### SUMMARY OF THE INVENTION

The current inventors, when measuring cellular avidity utilizing cells attached to a surface, were looking for means to collect the cells that remain bound to the attached cells after a cellular avidity measurement. Collecting the cells is of use e.g. in case it would be of interest to select the cells for further analysis or for further experimentation. More in particular, the inventors were working with target cells attached to a surface, with effector cells bound thereto, between which, if a specific interaction occurred, a synapse can be formed. The current inventors found that with cells attached to the surface, with effector cells bound thereto, that the cells can be obtained, e.g. by resuspension with a syringe and/or repeated pipetting. As shown in the examples, the inventors found that this method apparently resulted in a portion of the effector cells and target cells that were bound to each other were being separated. In addition, the cells that remained bound to each other could be substantially further separated by a simple enzymatic treatment, e.g. trypsin, resulting in single cells, which allows for further selection and subsequent use thereof. Without being bound by theory, such an enzymatic treatment allows to break synapses formed between effector cell and target cells resulting from a specific interaction.

Based on these results, the current inventors hypothesized that because the force applied during resuspension apparently resulted in further separation of cells, that such a step allows to provide a force which can further break target cell-effector cell bonds, because said force may exceed the maximum force applied during the cellular avidity measurement. Such a force may be referred to as a differential force that does not require cells attached to a surface. It is understood that attachment involves immobilization, i.e. by attachment of cells to a surface, cells are immobilized. Hence, phrased differently, a differential force is applied that does not require immobilization of either target cells or effector cells, e.g. by attachment to a surface. Hence, without being bound by theory, highly advantageously, such a differential force can be exerted after, or included in, a cellular avidity measurement that (further) breaks cell-cell bonds, in particular of aspecific interactions, whereas specific interactions that resulted in a synapse, and hence a highly strong interaction, are retained.

The current inventors realized that after a cellular avidity measurement, wherein cells were attached to a surface, in a subsequent step, a further force can be exerted which further breaks aspecific cell-cell interactions thereby resulting in specific cell-cell interactions, e.g. forming an immune synapse being retained. Moreover, the current inventors also realized that because the maximum force that can be exerted on the cell-cell interactions is no longer restricted by the strength of attachment of the cells to the surface, this allows for a method which no longer requires cells attached to a surface, providing a much more versatile method no longer complicated by the need to attach cells, and not restricted with regard to the maximum force that can be applied. This way, highly advantageously, attachment conditions do not need to be optimized and cellular avidity measurements can be improved as the force can be more easily selected to provide for an optimal window of separation.

Such improved means and methods are highly useful when it is for example desirable to select for and identify candidate effector cells that can bind to target cells and/or identify and/or analyse candidate compounds for modulating cellular avidity of effector cells to target cells. This is in particular highly useful in methods in which cellular avidity is used as a means, e.g. by applying a force to cells of interest that interact with target cells. In such cellular avidity methods, it is highly desirable and important to have the ability to differentiate well between specific binding of cells of interest and background binding of control cells to target cells. Hence, there is need in the art to provide for means and methods that may enlarge the window between positive and negative binding such that differentiation in cellular avidity between various cells of interest can be much improved, and/or cells of interest can be more efficiently identified.

Hence, in one embodiment, the current invention provides for a method for providing effector cells bound with target cells via a synapse comprising the steps of:
providing target cells;
providing effector cells;
contacting the target cells with the effector cells and allowing the cells to interact and form a synapse;
applying a force such that aspecific cell-cell bonds are broken and synapses are retained; thereby providing cells substantially comprised of target cells, effector cells, and effector cells bound with target cells via a synapse;
optionally, separating the effector cells bound with target cells via a synapse from the target cells and from the effector cells.

Subsequently, the number of effector cells bound with target cells via a synapse can be determined, and, optionally, the number of effector cells and/or target cells, and, a cellular avidity score can be determined based on the number of effector cells bound with target cells via a synapse.

It is understood that the target cells (or the effector cells), may also have been bound to a surface, such as depicted in FIG. 2, prior to collecting the cells. It is also understood that after the contacting and interaction step, the cells are collected and it may be that the collection step includes applying a force at the same time (e.g. via repeated pipetting or the like), such as described in the examples herein. The force may also be exerted directly after the contacting and interaction step without requiring collecting the cells, e.g. in case cells are held in a chamber and e.g. a sonification force is exerted to the cells held in the chamber.

### DESCRIPTION OF THE FIGURES

FIG. 1. Schematic showing target cells and effector cells and cellular avidity involving cells attached to a surface. Target cells (2) are provided on a surface (1), which as depicted is in this case a flat surface. The target cell expresses ligands and receptors, likewise the effector cell (5) to be targeting the target cell expresses ligands and receptors as well. Although in the figure the target cells are shown attached to a surface, the effector cells may be attached to the surface instead. Ligands and receptors can interact thereby forming a cell-cell bond. A specific ligand-receptor interaction, e.g. an antigen (3)-CAR (4) interaction, can be the driving force for the forming of a cell-cell bond with multiple ligand-receptor interactions combined ultimately resulting in strong cell-cell binding, e.g. a synapse. To rupture a cell-cell bond, a force (6) can be exerted on the effector cell (5) away from the target cell (2), which can be in the z-axis direction, e.g. when the flat surface is defined as being in the x-y plane. Alternatively, the force can also be applied in the x or y-axis direction. When the cell-cell bond is ruptured, the cell moves away from the cell surface and/or the target cell and this event can be detected and/or detached cells can be collected and quantified and/or further analysed, e.g. to determine cellular avidity.

FIG. 2. Schematic outlining the forces which play a role in cellular avidity measurements involving cells attached to a surface. Cells are provided attached to a surface. In the scenario depicted, a force Fₘ is exerted away, i.e. perpendicular, from the surface. The force, Fₘ, that can be exerted is restricted by the force at which the cells attached to the surface will detach therefrom (i.e. F_{c}), which means that Fₘ<F_{c}. With regard to a cellular avidity method, the forces that are required for cells (depicted as white cells) that have interacted with the attached cells (depicted as grey cells) to move away from the attached cells determine the outcome of a cellular avidity measurement. Ideally, the force required to have aspecifically bound cells move away, i.e. Fₐ, is lower than F_{c}, and the force required to have specifically bound cells that formed e.g. an immune synapse, i.e. F_{b}, is higher than Fₘ and Fₐ. In case aspecifically bound cells require a force Fₐ which is larger than the applied force Fₘ (and F_{c}), such aspecifically bound cells will remain and may confound cellular avidity measurements. Hence, cellular avidity measurements utilizing attached cells are restricted with regard to the maximum force that can be applied as those should not exceed F_{c}. As often occurs in cellular avidity measurements, as exemplified e.g. by background binding observed with control measurements, Fₐ for a portion of the cells can be larger than F_{c} and/or Fₘ, resulting in retaining at least part of the cells bound aspecifically at the end of an applied force, e.g. a force ramp. This means that bound cells at the end of a cellular avidity measurement, e.g. with an applied force ramp, often have either a synapse or are bound aspecifically to the cells attached to the surface.

FIG. 3. In a cellular avidity measurement with cells attached to a surface (depicted as grey cells), cells (depicted as white cells), are interacted with the attached cells to bind therewith, e.g. utilizing target cells expressing an antigen and effector cells with a CAR against the antigen. After a defined incubation, a force Fₘ is applied away from the attached cells. This results in cells detaching therefrom, either because they did not bind to the cells attached to the surface or because the binding strength was not strong enough, e.g. when aspecifically bound. Cells that remain were sufficiently strongly bound to the attached cells. These cells can subsequently be resuspended and a differential force, i.e. Fₙ is applied. Such a subsequently applied force may be larger than Fₘ. This force subsequently may further break cell-cell bonds, preferably aspecific cell-cell bonds, as these bonds are less strong when compared with synapse bonds. The cell suspension may be subsequently analysed and/or sorted with regard to singlets (either white or grey cells) and doublets (grey cell bound with white cell), which numbers may be used to provide a cellular avidity score, and which sorted cells may be subjected to further analysis.

FIG. 4. A cellular avidity measurement which does not require cells attached to a surface. In this scenario, e.g. target cells (depicted as grey cells) are provided and effector cells (depicted as white cells), and these are incubated for a defined period. After said incubation, cells are resuspended and a differential force, Fₙ, is applied. Such an applied force may be larger than typically used when cells are attached (Fₘ). This force may break cell-cell bonds, preferably aspecific cell-cell bonds as these bonds are less strong when compared with synapse bonds. The cell suspension may be subsequently analysed with regard to singlets (either white or grey cells) and doublets (grey cell bound with white cell), which numbers may be used to provide a cellular avidity score. When applying shear flow with repeated pipetting as depicted, as long as the differential force Fₙ exerted is larger than Fₐ (see FIG. 2 for explanation of Fₐ), i.e. the differential force that breaks aspecific cell-cell bonds, and of course smaller than F_{b} (see FIG. 2 for explanation of F_{b}), the cell-cell bonds that remain are substantially cell-cell bonds with a synapse.

FIG. 5. Schematic showing resuspension of cells obtained after applying a force on cells away from cells attached to a surface. After the force has been applied, (a), the cells can be collected, e.g. via resuspension, by forceful flushing causing high shear force, or other mechanical means (b). The obtained cells may be forced e.g. through a nozzle (c), which induces a differential force on cells bound to each other, i.e. the force exerted on the white cell is higher (F_{q}) than the force on the grey cell (Fᵣ), the net force exerted being F_{q}−Fᵣ=Fₛ. If the net force is large enough to break a cell-cell bond, the cell-cell bond will rupture. This can result in substantially aspecific cell-cell bonds to break, while cell-cell synapse bonds remain (d). In such a process, the same principles as depicted in FIG. 1 apply, the difference being that the differential force that is exerted is not restricted by the strength of the attachment of the cells to the surface, nor is it required to have the cells attached. This means that the net force Fₛ applied can be larger than the force that would be required to detach cells attached to the surface F_{c} (see FIG. 1).

FIG. 6. Scheme outlining fractions and calculations for a cellular avidity score. First target cells (M) and effector cells (N) are provided and allowed to interact. After the interaction step, single cells (N(i) and M(i)) of effector cells and target cells are obtained, and doublets are obtained as well, with the number of effector cells and target cells involved in cell-cell bonds (which can be with a synapse or can be an aspecific bond) being equal (N(ii) and M(ii), respectively, N(ii)=M(ii)). After a force in accordance with the invention has been exerted, single cells are obtained (M(iii) and N(iii)) and doublet cells, provided the force exerted allowed to substantially break aspecific cell-cell bonds, obtained consist substantially of cell-cell bonds in which a synapse was formed (again, with the number of effector cells and target cells involved therein being equal (N(iv) and M(iv), respectively, N(iv)=M(iv)). Finally, examples, which are non-exhaustive, of cellular avidity (CA) ratios that can be calculated are listed.

FIG. 7. FACS analysis of resuspended fractions treated with trypsin or with a PBS control. A) plots event counts versus FITC-A intensity for PBS control, and in B), for the population of the gated FITC-A counts depicted in A), FITC-A intensity was plotted against the intensity of the PB450 channel. Likewise, C) plots event counts versus FITC-A intensity for the trypsin treated fraction, and in D), for the population of the gated FITC-A counts depicted in C), FITC-A intensity was plotted against the intensity of the PB450 channel.

### DETAILED DESCRIPTION

As said, the current invention provides for improved means and methods for providing effector cells bound with target cells via a synapse. Means and methods in accordance with the invention are highly useful e.g. for determining cellular avidity or for sorting, selection and/or screening methods, e.g. aiming to find suitable receptors that are of use for effector cells or to find suitable effector cells, of use in treatments aimed at treating cancer, infectious disease, or the like.

In one embodiment, a method is provided for providing effector cells bound with target cells via a synapse comprising the steps of:
providing target cells;
providing effector cells;
contacting the target cells with the effector cells and allowing the cells to interact and form a synapse;
applying a force such that aspecific cell-cell bonds are broken and synapses are retained; thereby providing cells substantially comprised of target cells, effector cells, and effector cells bound with target cells via a synapse;
optionally, separating the effector cells bound with target cells via a synapse from the target cells and from the effector cells.

Hence, in the methods as described herein, effector cells carrying a receptor can be provided. In accordance with the invention, effector cells carrying a receptor include effector cells of the immune system that can exert an effect, via the receptor. For example, a T cell carrying a T cell receptor can bind an antigen on a cancer cell, upon which it can e.g. exert a cytotoxic effect and kill the target cell. Effector cells can be derived from nature, e.g. obtained from a host, and can also include genetically modified cells wherein e.g. a receptor in particular useful is provided to an effector cell.

Also, target cells are provided. Target cells in accordance with the invention are the cells on which the effector cells are to exert an effect, e.g. bind therewith and trigger an immune reaction thereto. Target cells include cancer cells presenting an antigen. An antigen may be presented by MHC, i.e. HLA in humans, which are specialized receptors that present peptides e.g. derived from digested proteins expressed by the cell (e.g. usually 8-11 amino acids in length for MHCI). An antigen may also be a protein or other biomolecule that is presented on the surface of a cell, e.g. epidermal growth factor receptors or checkpoint proteins, which in the case of cancer cells are overexpressed therewith providing a differentiating feature when compared with non-diseased cells. Target cells may also include cells expressing auto-antigens, e.g. known to be involved in autoimmune diseases or cells infected with a pathogen, e.g. a virus.

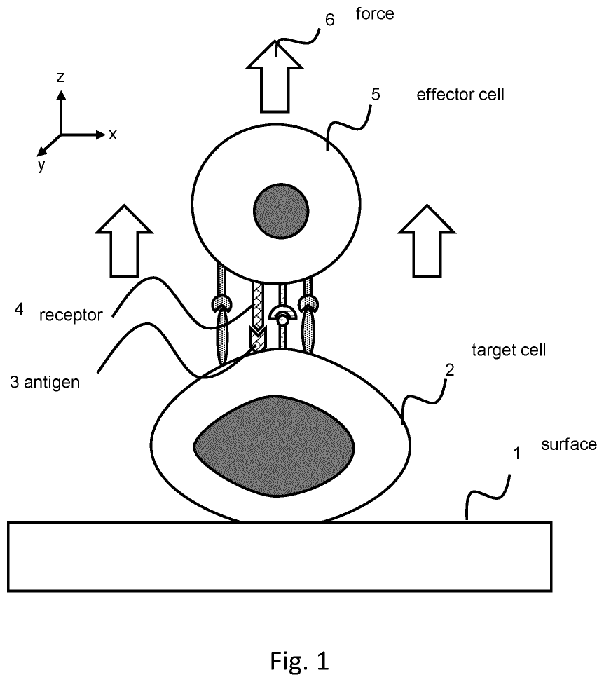

Next, the target cells and effector cells are brought into contact allowing the cells to interact and form a synapse. In this step, effector cells are contacted with the target cells to allow these cells to interact with each other such that the effector cells and target cells will have sufficient time in each other's proximity. The effector cells interact with the target cells and form a bond, such as a synapse, with a target cell (see i.a. FIG. 1). It is understood that a cell-cell interaction may not always result in a cell-cell bond, which can be a synapse or aspecific bond, the contacting step is such that cell-cell bonds can be formed and appropriate conditions therefore are selected. It is to be understood that because the conditions are selected such that a synapse can be formed, this necessarily implies that aspecific cell-cell bonds are allowed to be formed at the same time. Effector cells and target cells may be mixed in an appropriate volume and allowed to settle under gravity force in a holder. One may also first layer target cells and subsequently provide effector cells by e.g. gently dispersing these cells in the medium such that effector cells settle down on the layer of target cells. One can also do the reverse, first layer effector cells and subsequently disperse target cells in the medium.

It is understood that a synapse is a specialized structure that forms when the plasma membranes of two cells come into close proximity to transmit signals. Cells of the immune system form synapses that are essential for cell activation and function. Lymphocytes such as T cells, B cells and natural killer (NK) cells form synapses that can be referred to as immunological synapses. Such a synapse typically forms between immune effector cells and target cells, e.g. cells presenting an antigen. A non-limiting example is e.g. a T cell or a CAR-T cell (as an effector cell) and a cancer cell (as a target cell). The formation of synapses between e.g. an effector cell and a target cell, for example an APC (antigen presenting cell), is a hall-mark event and signals the presence of specific interactions (i.e. the specific interaction between, for example, a TCR or CAR and an antigen recognized thereby) between the effector cell and the target cell that are involved in the formation of such immunological synapses. It is understood that when synapses are allowed to form, this implies that aspecific cell-cell bonds are allowed to form as well.

In any case, after the contacting step, allowing for cell-cell bond formation including synapses, which contacting step can be well controlled and defined to allow for reproducibility between different methods performed, a differential force is exerted. It is understood that any suitable force may be exerted that allows to break cell-cell bonds, such as repeated pipetting, sonication, vibration, shear flow, or the like. In any case, the strength of the force that is exerted is selected such that aspecific cell-cell bonds are substantially broken, and such that cell-cell bonds, that can be characterized as a synapse, are substantially retained. As shown in the example section, resuspension utilizing a syringe or repeated pipetting in itself already exerts a force, i.e. a shear force, with which aspecific cell-cell bonds can break and with which synapses that have formed, can retain. Hence, after the force has been applied, the cells may be provided as a suspension. Of course, any method of applying a suitable force may suffice. Of course, because as opposed to the scenario wherein cells are attached to a surface, there may not be one direction of the force exerted, but the maximum force exerted can be controlled and by repeating the action, e.g. in the case of repeated pipetting or the like, one can ensure that all cell-cell bonds are subjected to a defined maximum force. Likewise, the same can apply to sonication force, and appropriate settings and conditions can be selected to apply a suitable controllable force in accordance with the invention. However, as the cells are not attached, or do not require to be attached, the force that is exerted is selected to be not a centrifugation force in one direction, or an acoustic force in a direction away from attached cells.

As it is understood that the force to be applied includes a differential force which does not require either of the target cells or effector cells to be attached, phrased differently, methods as provided in accordance with the invention, include methods for providing effector cells bound with target cells via a synapse comprising the steps of:
providing target cells;
providing effector cells;
contacting the target cells with the effector cells and allowing the cells to interact and form a synapse;
applying a differential force that does not require attachment to a surface of target cells or effector cells, such that aspecific cell-cell bonds are broken and synapses are retained; thereby providing cells substantially comprised of target cells, effector cells, and effector cells bound with target cells via a synapse; and wherein optionally, separating the effector cells bound with target cells via a synapse from the target cells and from the effector cells.

It is understood that it is not required to break all aspecific cell-cell bonds, which may break more often with a lower exerted force, and have all specific cell-cell bonds, which may break more often with a higher exerted, retained. Hence, one can apply a similar differential force, i.e. with regard to the amount of force exerted, as is current common practice with e.g. a device from LUMICKS (e.g. a selected force in the z-Movi® device), or even less. This is because in the current invention it may be highly advantageous to not have to attach cells to a surface, which can pose problems. For example, specific culturing conditions and/or surface coatings for attachment of cells may be challenging to define for certain cells. By avoiding the requirement of attachment, this may be circumvented.

In any case, after a force has been applied such that aspecific cell bonds are broken and synapses are retained; thereby cells are provided substantially comprised of target cells, effector cells, and effector cells bound with target cells via a synapse. It is understood that the target cells and effector cells obtained as referred to are unbound cells. Of course, effector cells bound with target cells via aspecific cell-cell bonds may be provided as well, as this may depend, as outlined above, on the strength of the force that is applied, with the larger the force, the less aspecific cell-cell bonds may remain. For example, the number of aspecific cell-cell bonds that are left, are less than 50%, less than 40%, less than 30%, or less than 20%. The number of aspecific cell bonds are between 0 and 50%, between 1 to 50%, between 1 and 40%, between 1 to 30%, between 1 to 20%, or between 1 and 10%. This percentage is relative to the total number of cell-cell bonds after the force has been applied. Hence, conversely, as the number of aspecific cell-cell bonds and the number of synapses combined add up to 100%, the number of cell-cell bonds with a synapse, or a marker associated with forming a synapse, are more than 50%, more than 60%, more than 70%, or more than 80%. The number of cell-cell bonds with a synapse are between 50 to 100%, between 60 and 100%, between 70 to 100%, between 80 to 100%, or between 90 and 100%. It may be preferred to have a high percentage of cell-cell bonds with a synapse and a low percentage of aspecific cell-cell bonds.

Subsequently, and optionally, the effector cells bound with target cells, having a synapse, which may also include aspecific cell-cell bonds as well, are separated from the target cells and from the effector cells. It is understood that this separation involves the separation of so-called doublets from singlets, i.e. unbound target cells or unbound effector cells may be referred to as singlets, consisting of single cells, and a target cell bound with an effector cell may be referred to as a doublet, i.e. consisting of two cells. The terms singlet and doublet are terms commonly used in flow cytometry and fluorescent activated cell sorting analysis or the like, and singlet cells and doublet cells can easily be separated from each other utilizing FACS, or the like, such as described in the example section. Separating the target cells and effector cells from target cells bound to effector cells can be useful as it may prevent further interaction, as the defined interaction step can be selected as representing a meaningful biological property of e.g. the effector cell. Hence, performing a separation step after applying a force can be useful, as it allows to separate unbound effector cells from bound effector cells, when e.g. the effector cells are to be subjected to further analysis or used in subsequent experiments.

In another embodiment a method is provided for providing effector cells bound with target cells via a synapse comprising the steps of:
providing target cells;
providing effector cells;
contacting the target cells with the effector cells and allowing the cells to interact and form a synapse;
applying a force, allowing at least for aspecific cell-cell bonds to be broken, thereby providing cells substantially comprised of target cells, effector cells, and effector cells bound with target cells via a synapse;
optionally, separating the effector cells bound with target cells from the unbound target cells and from the unbound effector cells.

In yet another embodiment a method is provided for providing effector cells bound with target cells via a synapse comprising the steps of:
providing target cells;
providing effector cells;
contacting the target cells with the effector cells and allowing the cells to interact and form a synapse;
resuspending the cells and applying a force, allowing at least for aspecific cell-cell bonds to be broken, thereby providing cells substantially comprised of target cells, effector cells, and effector cells bound with target cells via a synapse;
optionally, separating the effector cells bound with target cells from the unbound target cells and from the unbound effector cells.

In yet another further embodiment a method is provided for providing effector cells bound with target cells via a synapse comprising the steps of:
providing target cells;
providing effector cells;
contacting the target cells with the effector cells and allowing the cells to interact and form a synapse;
applying a force, thereby providing cells substantially comprised of target cells, effector cells, and effector cells bound with target cells via a synapse;
optionally, separating the effector cells bound with target cells from the unbound target cells and from the unbound effector cells.

The steps of the methods as described herein and in accordance with the invention may be highly useful for determining cellular avidity. Hence, in another embodiment, a method is provided for determining cellular avidity, comprising the steps of:
providing target cells;
providing effector cells;
contacting the target cells with the effector cells and allowing the cells to interact and form a synapse;
applying a force such that aspecific cell-cell bonds are broken and synapses are retained; thereby providing a suspension of cells substantially comprised of target cells, effector cells, and effector cells bound with target cells via a synapse;
optionally, separating the effector cells bound with target cells having a synapse from the target cells and from the effector cells;
determining the number of effector cells bound with target cells via a synapse, and, optionally, the number of effector cells and/or target cells; and
determining a cellular avidity score based on the number of effector cells bound with target cells via a synapse.

In another embodiment, a method is provided for determining cellular avidity, comprising the steps of:
providing target cells;
providing effector cells;
contacting the target cells with the effector cells and allowing the cells to interact and form a synapse;
applying a force such that aspecific cell-cell bonds are substantially broken and synapses are substantially retained; thereby providing a suspension of cells substantially comprised of target cells, effector cells, and effector cells bound with target cells via a synapse;
optionally, separating the effector cells bound with target cells having a synapse from the target cells and from the effector cells;
determining the number of effector cells bound with target cells via a synapse, and, optionally, the number of effector cells and/or target cells; and
determining a cellular avidity score based on the number of effector cells bound with target cells via a synapse.

In another embodiment, a method is provided for determining cellular avidity, comprising the steps of:
providing target cells;
providing effector cells;
contacting the target cells with the effector cells and allowing the cells to interact and form a synapse;
applying a force allowing for at least aspecific cell-cell bonds to be broken;
thereby providing a suspension of cells substantially comprised of target cells, effector cells, and effector cells bound with target cells via a synapse;
optionally, separating the effector cells bound with target cells having a synapse from the target cells and from the effector cells;
determining the number of effector cells bound with target cells via a synapse, and, optionally, the number of effector cells and/or target cells; and
determining a cellular avidity score based on the number of effector cells bound with target cells via a synapse.

In yet another embodiment, a method is provided for determining cellular avidity, comprising the steps of:
providing target cells;
providing effector cells;
contacting the target cells with the effector cells and allowing the cells to interact and form a synapse;
applying a force to thereby provide a suspension of cells substantially comprised of target cells, effector cells, and effector cells bound with target cells via a synapse;
optionally, separating the effector cells bound with target cells having a synapse from the target cells and from the effector cells;
determining the number of effector cells bound with target cells via a synapse, and, optionally, the number of effector cells and/or target cells; and
determining a cellular avidity score based on the number of effector cells bound with target cells via a synapse.

It is understood that a force is selected to highly preferably provide for a suspension of cells substantially comprised of target cells, effector cells, and effector cells bound with target cells via a synapse. However, it may not necessarily be required to select such a force to allow the effector cells bound with target cells to substantially consist of effector cells bound with target cells via a synapse. A force may also be selected and applied to provide for target cells, effector cells, and effector cells bound with target cells instead. Just counting doublets observed that remain after the force has been exerted, and singlets of effector cells and/or target cells. It may also be useful to count the number of doublets (in relation to e.g. the number of total effector cells) for both cells of interest and for control cells of cells of interest, and subsequently compare the cellular avidity scores determined. In another embodiment, a method is thus provided for determining cellular avidity, comprising the steps of:
providing target cells;
providing effector cells;
contacting the target cells with the effector cells and allowing the cells to interact and form a synapse;
applying a force such that aspecific cell-cell bonds are substantially broken and synapses are substantially retained; thereby providing target cells, effector cells, and effector cells bound with target cells;
optionally, separating the effector cells bound with target from the target cells and from the effector cells;
determining the number of effector cells bound with target cells, and, optionally, the number of effector cells and/or target cells; and
determining a cellular avidity score based on the number of effector cells bound with target cells.

In yet another embodiment, a method is provided for determining cellular avidity, comprising the steps of:
providing target cells;
providing effector cells;
contacting the target cells with the effector cells and allowing the cells to interact and form a synapse;
applying a force allowing for at least aspecific cell-cell bonds to be broken to thereby provide target cells, effector cells, and effector cells bound with target cells;
optionally, separating the effector cells bound with target from the target cells and from the effector cells;
determining the number of effector cells bound with target cells, and, optionally, the number of effector cells and/or target cells; and
determining a cellular avidity score based on the number of effector cells bound with target cells.

It may also be useful to count the number of doublets (i.e. number of effector cells bound with target cells, and in relation to e.g. the number of total effector cells) for both cells of interest (i.e. effector cells, e.g. targeting a cancer antigen) and for control cells of cells of interest (a proper control for the effector cells, e.g. targeting a control antigen), and subsequently compare the cellular avidity scores determined.

Methods for determining cellular avidity, or a cellular avidity score, in the art involve measurements of the amount of cells that form a cell-cell bond, which, in the case of effector cell-target cell bonds, preferably involves immune synapses as those are of interest, e.g. when selecting suitable receptor candidates and/or suitable effector cells. Hence, determining cellular avidity includes at least the number of effector cells bound with target cells, of which highly preferably a substantial portion involves effector cells bound with target cells via a synapse. Of course, suitable highly stringent conditions may be selected such that a high proportion of the cell-cell bonds formed after the interaction step involve immune synapses, but this may not necessarily be required. Suitable conditions and/or forces may be selected e.g. before experiments are to be performed, e.g. by utilizing control cells. Control cells may be provided of either the effector cells or of the target cells. For example, in case effector control cells are provided, these may be control cells that do not have a receptor that is to bind to the target cells. In case target control cells are provided, these may be target cells that do not express an antigen which the effector cell is to target.

In one embodiment, a method is provided of selecting a force to be applied, for breaking cell-cell bonds between target cells and effector cells, comprising the steps of:
a) providing target cells;
b) providing effector cells
c) providing effector control cells or target control cells;
d) contacting the effector control cells with the target cells; or the effector cells with the target control cells and allow the cells to interact and form cell-cell bonds;
e) applying a first force;
f) determine the percentage of control cells contacted in step d) that remain bound;
g) perform steps d), e) and f) with the effector cells and the target cells;
h) optionally, perform steps d), e) and f), and g) with one or more further forces;
i) select the force to be applied from the first, and optional further forces, by comparing, with the same force applied, the percentages of target control cells or effector control cells that remained bound; and of control cells and effector cells that form a cell-cell bond.

Of course, instead of percentages, one could equally well determine another relative measurement representing cell-cell bonds that have formed between cells. It is understood that control cells refer to basically the same cells as the cells of interest, i.e. target cells or effector cells, but these cells e.g. do not express a ligand/antigen or receptor, and/or may express e.g. a variant thereof which is not functional, i.e. with regard to allowing for a specific cell-cell bond formation that can result in substantial formation of an immune synapse. It may be preferred that about 25% or less, more preferably about 15% or less, most preferably about 10% or less, of the control cells remain bound after the force has been exerted (i.e. control cells of target cells bound to effector cells or control cells of effector cells bound to target cells). Of course, conditions may preferably be selected such that not 100% of target cells and effector cells are bound after the force has been exerted in order to allow to detect differentiation between different effector cells. For example, in a scenario, wherein of a particular cell of interest, e.g. a defined CAR-T, cellular avidities are to be subsequently selected which are to be improved and/or reduced with regard to cellular avidity properties, in order to provide for a range of different cellular avidities, it may be desirable to select for about 50/60% binding of the defined CAR-T, and subsequently test the modified versions thereof which allows to detect improvements (i.e. exceeding 50/60%) vs. reductions (i.e. below 50/60%).

Preferably, the percentage difference of a specific interaction between effector cells and target cells as compared with a control interaction (e.g. of an effector control cell and the target cell) is at least 30%, or more. Preferably, a force may be selected with the largest difference between the percentages. Preferably, the percentage of control cells remaining bound is respectively 15% or below, and of the non-control cells, i.e. effector cells, this preferably is in the range of 85% or higher. This percentage being relative to the total number of effector (control) cells. More preferably, in a scenario wherein e.g. a force is to be selected for sorting purposes and the like, it may be desirable to have no control cells remaining attached, or at least a low percentage, such as 5% or less, 4% or less, or 3% or less, of the total number of control cells. With any other unit or cellular avidity score determined, optimal differentiation windows can likewise be selected. In any case, by varying the forces to be applied, suitable and advantageous conditions for subsequent means and methods relying on cellular avidity can be advantageously selected and used in the methods of the invention as outlined herein. Suitable conditions include appropriate cell culture media and culture conditions, as well as incubation time, and force applied. Of course, when e.g. comparing cellular avidity experiments and/or determined cellular avidity scores, such conditions are preferably well controlled to allow appropriate comparisons and therewith avoiding undesirable variation, as is common practice and understood by the skilled person.

Of course, it may not be necessary to use control cells for determining and/or selecting a suitable force, in addition to using effector cells and target cells. Nor may it be necessary to use effector cells and target cells, in addition to using control cells for determining and/or selecting a suitable force. One may also solely use control cells of target cells and effector cells, or solely use control cells of effector cells and target cells, and determine the minimal force to allow for a suitable percentage of cell-cell bonds to remain. Conversely, one may also solely use target cells and effector cells and determine a maximal force that allows the cell-cell bonds associated with synapse formation to remain. However, in the latter case, it may be preferred to also assess cell viability, as exerting a force may have effect thereon. Hence, a force may be selected that allows for cell-cell bonds, and more preferably cell-cell bonds having a synapse, to substantially remain while retaining cell viability. Alternatively, a force may be selected that allows aspecific cell-cell bonds to substantially be broken while retaining cell viability. Means and methods to determine cell viability are well known in the art, and include e.g. trypan blue staining and the like.

It is understood that the above-mentioned percentages may be referred to as a cellular avidity score. These percentages can be calculated based on the determined number of cell-cell bonds that are detected, or the determined relative number, e.g. when of a fraction of a cell suspension the number of doublets and singlets, representing target cells bound to effector cells and unbound target cells and effector cells is determined. For example, the number of doublets divided by the number of effector cells initially provided (or proportionate fractions thereof), provides for a cellular avidity score. Also, the number of doublets may be divided by the number of singlet effector cells plus the number of doublets, to provide for a cellular avidity score. One may even envision that one provides only a (relative) number representing doublets, i.e. target cells bound to effector cells. For example, when conditions are well controlled, this number may itself be representative of cellular avidity, without the need to determine a ratio.

The target cells that are attached to the surface, for example attach as a monolayer. The monolayer preferably has a high confluency. The subsequent cells of interest (and/or control cells thereof) that are to interact with the target cells are preferably provided in a relatively low cell density as compared with the target cells, such that substantially all cells of interest (and control cells thereof) can interact with a target cell (there are more target cells per cell of interest). Such provides for advantageous controllable conditions when applying the force on the control cells or cells of interest.

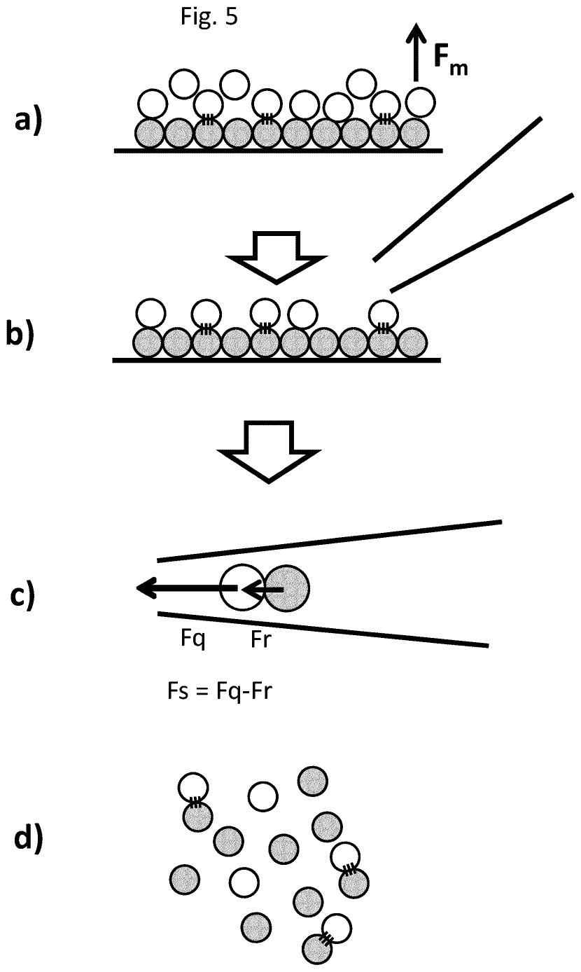

As is clear from the above, the type of force that is to be applied in accordance with the invention is a force capable of breaking cell-cell bonds, i.e. the force exerted causes cells bound to each other to have the cells move away from each other to such an extent that a cell-cell bond may break or rupture. A differential force means that the force on one cell differs from the force on the other cell with regard to direction of the force and/or the magnitude of the force, resulting in a net force allowing to break cell-cell bonds if the differential force exceeds the binding force. For example, when a target cell bound to an effector cell, a doublet, is forced through a nozzle, the closer to the throat of the nozzle, the faster the flow. This means that the first cell to enter the nozzle is subjected to a stronger acceleration than the cell lagging behind and the cells experience a differential force resulting in a net force which can result in cell-cell bond rupture, provided the force is large enough (see e.g. FIG. 5). As indicated above, the differential force that can be applied includes a shear force, e.g. such as can be applied utilizing repeated pipetting (repeated upwards and downwards flow of the sample) or flow through a nozzle. Other means and methods are known in the art with which shear forces can be applied to cells, e.g. flowing a cell suspension at a constant speed and bombarding these cells to a flat surface at a defined angle. Furthermore, forcing a cell suspension through a needle with a defined internal diameter and a defined force may provide for a well controllable shear force as well. The cell suspension may be subjected to several rounds of such process steps to ensure substantially all cell-cell bonds experience the maximum force that may be achieved with the process step. Such a process step allows for automation, enabling control and repeatability of the process, therewith controlling shear forces exerted. Suitable devices for breaking apart cell-cell bonds which are not synapses are known in the art (e.g. Zahniser et al., J. Histochem. Cytochem. 1979, 27 (1), 635-641). Also, by properly tuning the forces in a flow-cytometer normally used to measure cell deformations, such as e.g. described in Otto, et al. Nat. Methods 12, 199-202 (2015) suitable forces can be applied. Tuning can be achieved e.g. by changing the nozzle size or geometry and/or the flow speeds used. Other suitable devices known in the art may include for a vortex mixer, with which shear forces may be suitably applied as well. Accordingly, in one embodiment, the force applied involves a shear force.

In another embodiment, the force applied is an ultrasonic force. It is understood, as outlined above, that such ultrasonic forces are not forces such as applied e.g. in a device as available from LUMICKS, wherein the force is away from attached cells (e.g. such as in the LUMICKS z-Movi® Cell Avidity Analyzer, e.g. as used by Larson et al., Nature 604, 7906:1-8, Apr. 13, 2022). It is also understood that the ultrasonic force is selected such that cells are not lysed. Hence, appropriate ultrasonic forces can be applied to cells such that cell-cell bonds can be ruptured, which more preferably includes breaking aspecific cell-cell bonds and less preferably breaks specific cell-cell bonds in which an immune synapse is formed. Examples of using ultrasonic forces to break (aspecific) cell-cell bonds, are known in the art (e.g. as described in Buddy et al., Biomaterials Science: An Introduction to Materials in Medicine, 3^{rd} edition, 2013, Chapter II. 2.8, page 576; and Moore et al., Experimental Cell Research, Volume 65, Issue 1, 1971, pages 228-232).

In any case, suitable applied forces which are known in the art include e.g. a force in the range of 50 pN-10 nN, which said force is a net force exerted on one cell relative to the other cell, of two cells bound to each other. Which means the force is exerted on the cell-cell bond. In another embodiment, the force exerted on one of the two cells relative to the other cell is at least 50 pN, or at least 100 pN, or at least 200 pN. In another embodiment, the force exerted is at most 10 nN, at most 5 nN, at most 3 nM, at most 2 nM, or at most 1 nN. In yet another embodiment, the force is selected from the range of 1 pN-10 nN, from 100 pN-10 nN, from 500 pN-10 nN, from 1 nN-10 nN. In still a further embodiment, the force is selected from the range of 500 pN-5 nN, from 500 pN-4 pN, from 500 pN-3 pN. For example, a suitable amount of force that can be exerted between cells (e.g. such as in the z-Movi© device) can be selected to be in the range of 200 pN-3000 pN. Of course, these force ranges are known to be useful with cells attached to a surface, and the maximum force that may be selected may exceed 3000 pN as it is not required to have the cells attached to a surface in accordance with the invention.

Accordingly, as it is understood that the differential force to be applied includes a differential force which does not require either of the target cells or effector cells to be attached, methods as provided in accordance with the invention, include methods for providing effector cells bound with target cells via a synapse comprising the steps of:
providing target cells;
providing effector cells;
contacting the target cells with the effector cells and allowing the cells to interact and form a synapse;
applying a differential force in the range of 50 pN-10 nN that does not require attachment of target cells or effector cells;
thereby providing cells substantially comprised of target cells, effector cells, and effector cells bound with target cells via a synapse; and
optionally, separating the effector cells bound with target cells via a synapse from the target cells and from the effector cells.

In another embodiment, methods are provided in accordance with the invention for providing effector cells bound with target cells via a synapse comprising the steps of:
providing target cells;
providing effector cells;
contacting the target cells with the effector cells and allowing the cells to interact and form a synapse, wherein neither the target cells not the effector cells are attached to a surface;
applying a differential force in the range of 50 pN-10 nN, thereby providing cells substantially comprised of target cells, effector cells, and effector cells bound with target cells via a synapse; and
optionally, separating the effector cells bound with target cells via a synapse from the target cells and from the effector cells.

In yet another embodiment, methods are provided in accordance with the invention for providing effector cells bound with target cells comprising the steps of:
providing target cells;
providing effector cells;
contacting the target cells with the effector cells and allowing the cells to interact and form a cell-cell bond, wherein neither the target cells not the effector cells are attached to a surface;
applying a differential force in the range of 50 pN-10 nN, thereby providing a mixture of cells substantially comprised of target cells, effector cells, and effector cells bound with target cells; and
optionally, separating the effector cells bound with target cells from the target cells and from the effector cells.

Without being bound by theory, as is understood in accordance with the invention, the range of force that may break an aspecific cell-cell bond versus a specific cell-cell bond that forms a synapse differs. This difference can be to such an extent that the ranges of the required forces do not overlap. It is understood that some overlap may occur. Hence, the force that is selected, as outlined above, may allow for aspecific cell-cell bonds remaining and some specific cell-cell bonds that formed a synapse to break. In case there is substantially no overlap, a differential force can be selected, as outlined above, which allows substantially for aspecific cell-cell bonds to break, while substantially retaining specific cell-cell bonds that formed a synapse. In case there is no overlap, and ranges are sufficiently far apart, a differential force may be selected, as outlined above, which allows for aspecific cell-cell bonds to break while retaining specific cell-cell bonds that formed a synapse. Hence, as also outlined herein, the portion of cell-cell bonds that can be assessed or confirmed as consisting of a synapse, can be determined by determining the presence or absence of a marker associated with synapse formation. Assessing the presence or absence of a synapse may be useful in selecting an appropriate differential force that allows to (most) selectively break aspecific cell-cell bonds and retain cell-bonds associated with a synapse.

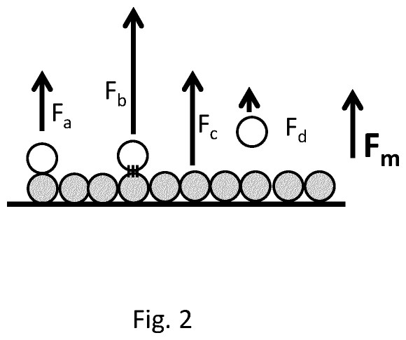

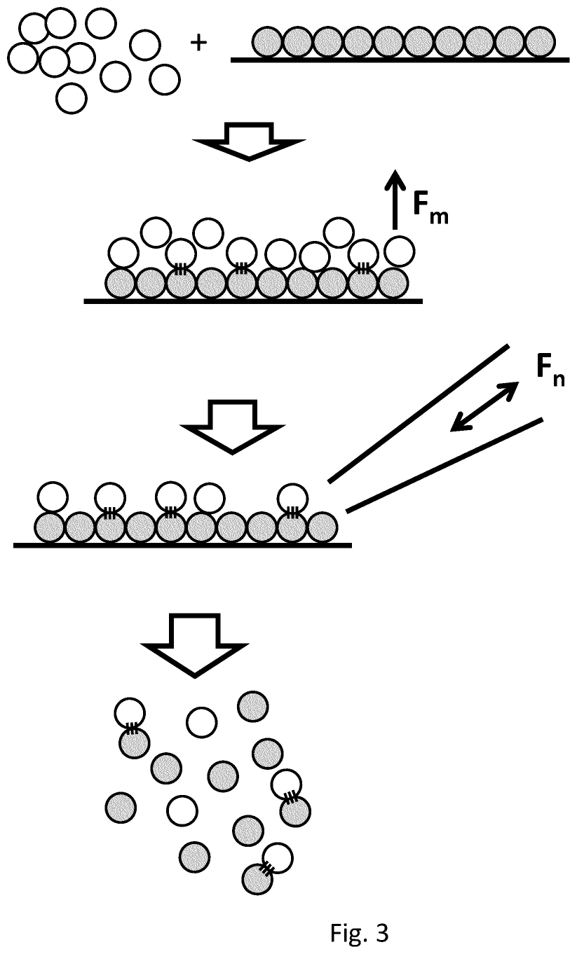

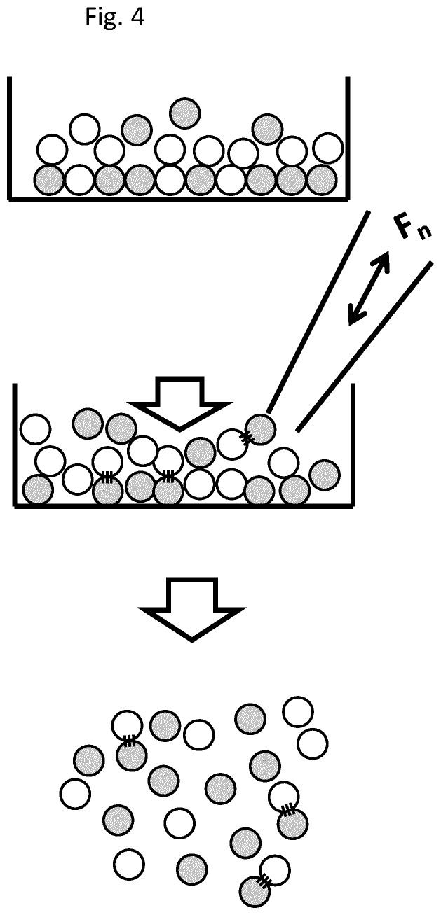

In another embodiment, in the methods in accordance with the invention, the target cells or the effector cells, are attached to a surface during the contacting step (see e.g. FIG. 2). As said, it may not be required to have the cells attached to a surface (e.g. as shown in FIG. 4), but it may still be advantageous to have the cells attached (e.g. as schematically depicted in FIG. 3), e.g. as it may allow to control the interaction between the target cells and effector cells. Once, the interacting step has been performed, and subsequently the force is to be applied, this subsequent force that may be applied may thus exceed the force required to detach the attached cells to the surface. Hence, the force that is applied to break cell-cell bonds may be selected to exceed the force required to detach cells when these are attached to a surface.

When a force is applied in a direction away from the attached cells, it is understood that in this step, the force applied may be perpendicular (in the direction of z-axis) to the surface (x,y) to which cells are attached, for example when a centrifugal force or acoustic force is applied. The force may also be lateral (in the direction of the x-axis or y-axis relative to the surface), for example when a shear force is applied. In any case, the force is applied and is controlled such that a defined force is exerted on the cells that interacted with the attached cells. It is understood that the force that is exerted on the cells interacting with attached cells is to be substantially equal, such can be achieved e.g. when using a flat surface. Other suitable surface shapes may be used (e.g. a tube with exerted concentrical force or laminar flow force in the direction of the length of the tube), as long as the force exerted can be substantially equal at a defined surface area to which cells are attached, such a surface shape may be contemplated. Any suitable force application method in a direction away may be contemplated in accordance with the invention in these embodiments. The force can be well controlled with acceleration-based methods of applying force such as centrifugation, and with shear flow and with acoustic force, as well, which are all suitable means for applying forces in a direction away from attached cells, but any other means of controllably causing a force on the cells attached to the target cells, thereby forcing them away from the target cells may be contemplated. Accordingly, in a further embodiment, the force that is to be applied in a direction away from attached cells is selected from is an acoustic force, a shear flow force or an acceleration force, such as a centrifugation force.

The cells may be attached to a plastic surface or a glass surface, such as a glass surface in a chip (i.a. as described in WO 2018/083193, and such as available from LUMICKS for the z-Movi® device). The cells that are attached to the surface, preferably are attached as a monolayer. The monolayer preferably is at high confluency. The subsequent cells that are to interact with the attached cells are preferably provided in a relatively low cell density as compared with the attached cells, such that substantially all of the cells provided can interact with the attached cells (there are more attached cells per cells subsequently provided). Such provides for advantageous controllable conditions of interaction.

In a further embodiment, when the target cells or effector cells are attached to the surface, after the contacting step, the cells that are not attached to the surface or not bound to the cells attached to the surface, are removed prior to applying the force as defined herein. This way, cells that did not have any interaction with the attached cells can easily be removed, which may be highly useful in subsequent analysis and/or for subsequent experiments.

In still a further embodiment, when the target cells or effector cells are attached to the surface, it may also be envisioned to first apply a force away from the cells attached to the surface, e.g. to determine cellular avidity (e.g. such as in the z-Movi© device), and subsequently apply the force as defined herein which may allow to substantially break aspecific cell bonds and substantially retain synapses. Different forces may be advantageously combined. For example, such combined methods may allow to provide for an indication of cell-cell bonds that are formed after a defined interaction and an indication as to the portion of cell-cell bonds that represents a specific interaction in which a synapse was formed. Such information may be highly informative and advantageous as well.

In another embodiment, in methods in accordance with the invention, an incremental force is applied and, optionally, fractions obtained from different forces are obtained, and wherein preferably the maximum force applied is such that aspecific cell bonds are substantially broken and synapses are substantially retained. In yet another embodiment, an incremental force is applied and, optionally, fractions obtained from different forces are obtained. It is understood that it may not be necessary to apply a maximum force to rupture or break synapses. Generating a cellular avidity curve, i.e. plotting e.g. (percentages or numbers of) doublets remaining vs. the force applied can be contemplated.

Once the force has been applied such that aspecific cell bonds are substantially broken and synapses are substantially retained; to thereby provide target cells, effector cells, and effector cells bound with target cells, subsequently, in another embodiment, effector cells bound with target cells via a synapse are separated from the target cells, preferably with trypsin. It is understood that in this embodiment, doublets comprising a target cell and an effector cell that remain may be further separated into a singlet target cell and a singlet effector cell, without exertion of a force, but rather by utilizing enzymatic cleavage of the bond between cells. Highly surprisingly, as shown in the example section, utilizing trypsin allows to cleave cell-cell bonds which, without being bound by theory, comprise a substantial amount of specific cell-cell bonds that formed a synapse. This way, e.g. effector cells may be obtained which may be further expanded, subjected to further analysis, or used in subsequent experiments. The use of trypsin to detach cells attached to a surface is widely known in the art and suitable trypsin solutions are commonly available. Hence, recommended conditions as provided by trypsin manufacturers may be applied in this step. It is understood that as often growth culture medium comprises a large amount of protein, it is preferred that cells are washed with medium with a low or no protein content in order to avoid quenching trypsin activity. Highly surprisingly, it was observed that under the conditions tested, trypsin cleaved specific cell-cell bonds which are involved synapse formation, while the cells remain viable as can be judged from FACS analysis, as shown in the example section, including the fact that cells remained fluorescently labelled.

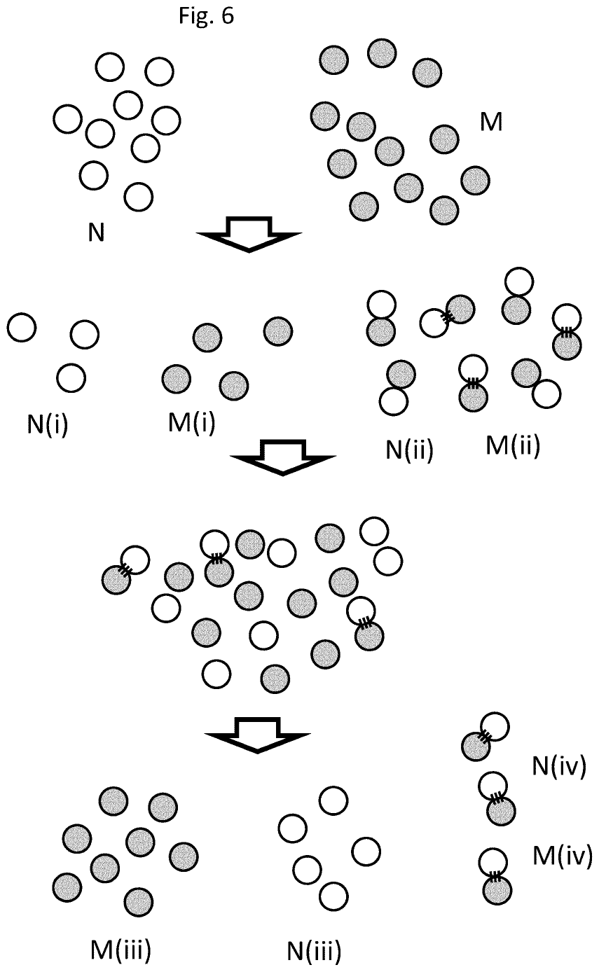

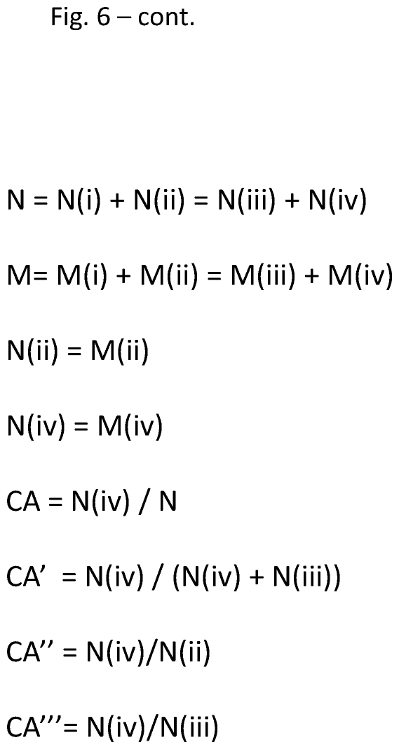

As said, based on the determined numbers of singlets and/or doublets, a cellular avidity score can be determined. For example, as shown and described in FIG. 6, determining the numbers of singlets and/or doublets, or representative portion thereof, in fractions obtained, exemplary cellular avidity scores can be determined. Hence, in a further embodiment, the cellular avidity score is determined by calculating the ratio of the number of effector cells remaining bound to target cells after the force has been exerted, (N(iv)), to the number of effector cells (N) that have interacted with the target cells in the contacting step; or by calculating the ratio of the number of effector cells remaining bound to target cells after the force has been exerted, (N(iv)), to the number of effector cells bound to the target cells after the interaction step (N(ii)); or by calculating the ratio of the number of effector cells remaining bound to target cells after the force has been exerted, (N(iv)), to the number of effector cells that are unbound after the force has been exerted (N(iii)). Of course, in case the force that is exerted allows to differentiate between aspecific cell-cell bonds and specific cell-cell bonds associated with synapse formation, one may refer to these calculations as the cellular avidity score as determined by calculating the ratio of the number of effector cells associated with synapse formation, (N(iv)), to the number of effector cells (N) that have interacted with the target cells in the contacting step; or by calculating the ratio of the number of effector cells associated with synapse formation, (N(iv)), to the number of effector cells bound to the target cells after the interaction step (N(ii)); or by calculating the ratio of the number of effector cells associated with synapse formation, (N(iv)), to the number of effector cells that are unbound after the force has been exerted (N(iii)). As is understood herein, the ratios determined may be based on absolute numbers, or may be calculated based on fractions obtained and analysed. One may represent the ratios calculated as a number or as a percentage.

Further means to enable collection or identifying an effector cell or target cell include means and methods wherein cells are provided with a photoactive label, as exemplified in the example section herein, or with a photoactivatable label that may be subsequently activated by illumination with light of a suitable wavelength only in a well-defined interaction region of the device (e.g. in a flow channel or in a defined region of a cell chamber) to photoactivate and/or switch the dye. Subsequently, the cells can be sorted for example using fluorescence activated cell sorting (FACS) and only those cells which are activated may further be used according to the methods described herein, thereby obtaining the cells on which defined forces have been exerted. This may for example be highly useful for collecting effector cells provided with a photoactive or photoactivatable label that remained attached to the target cells after the force was exerted and after these doublets were separated from singlets, and optionally separated from e.g. the target cells via a trypsin treatment. Alternatively, singlets and doublets may also be differentiated with light scatter signals in cell sorting devices, not necessarily requiring fluorescent signals.

Highly advantageously, as indicated herein, the force required to rupture a cell-cell bond which involves a specific interaction and synapse formation in general may be relatively large as compared with the force required to rupture an aspecific interaction. Nevertheless, a force may be selected to break cell-cell bonds which may leave a substantial portion of the aspecific cell-cell bonds intact, i.e. of the effector cells bound with target cells. Hence, in such a scenario, it may be highly advantageous to determine in effector cells bound with target cells, the presence of a marker associated with synapse formation in order to differentiate between specific and aspecific interactions. Conversely, it may also be advantageous to merely as validation or confirmation of the force selected to break cell-cell bonds that are to allow synapses formed to remain intact, to determine the presence or absence of a marker associated with synapse formation. The marker may be detected preferably after the force has been exerted, because the force that is applied usually does not exceed the force required to break an immune synapse. In some embodiments, e.g. with high exerted forces, it may be useful to determine synapses prior to applying the force and/or after applying the force. Of course, in these latter embodiments, the methods for detection of synapses highly preferably allow the cells to remain intact and alive. Detecting synapses before applying the force, and subsequently comparing with detecting synapses after the force has been applied may e.g. allow to confirm that the applied force did not rupture synapses formed.

In the case of a T cell, a synapse can be formed between the lymphocyte and antigen-presenting cells (APCs) during the recognition of the peptide antigen-major histocompatibility complex (pMHC) ligand by the T cell antigen receptor (TCR). The TCR and pMHC are both membrane-bound, so the TCR will only be triggered by its ligand at the interface between T cells and APCs. A synapse can be observed at the T cell-APC interface as concentric rings by confocal microscopy, often referred to as “bull's eye” (Huppa, J. B., & Davis, M. M. (2003), T cell-antigen recognition and the immunological synapse. Nature Reviews Immunology, 3(12), 973-983). These rings were named the central, peripheral, and distal supramolecular activation cluster (i.e. respectively cSMAC, pSMAC and dSMAC). The TCR has been reported to be present in the cSMAC, whereas other lymphocyte specific proteins such as lymphocyte function-associated antigen-1 (LFA-1), are integrated into the pSMAC ring that surrounds the TCR. The formation of this ringed structure (“bull's eye”) is however not universal and other formations such as “multifocal immunological synapses” between T cells and dendritic cells, or the like, have been described.

The immunological synapse can be considered to be any structure formed at the interface resulting from a functional and specific effector-target cell interaction, such as for example T cell-APC contacts. Markers associated with effector cells and synapse formation include one or more of CD43, CD44, CD45, LFA-1, Talin, F-actin, ZAP70, CD2, CD4, CD8, CD3, CD28, PD-1, ICOS, and TCR. Markers of target cells include one or more of ICAM-1 (associates with LFA-1), CD48/58 (interacting with CD2) CD80/CD86 (interacting with CTLA-4 and CD28), PDL1/PDL2 (associating with PD-1), and MHC presenting the antigen (that specifically interacts with the TCR). These markers, and concentrations thereof, may be detected e.g. with fluorescent labels at the interface between effector cell and target cell, or intracellularly in close proximity to the synaptic interface.

For example, markers of effector cells that have been associated with dSMAC are CD43, CD44, CD45. The effector cell markers LFA-1, Talin, F-actin, CD2, CD4 and CD8 have been associated with pSMAC. The markers CD3, CD28, PD-1, ICOS, and TCR of effector cells have been associated with cSMAC. Markers that have been associated with a synapse on a target cell are ICAM-1 (associates with LFA-1), CD48/58 (interacting with CD2) CD80/CD86 (interacting with CTLA-4 and CD28), and PDL1/PDL2 (associating with PD-1), and of course an MHC presenting the antigen (that specifically interacts with the TCR). These markers have been shown to be associated with synapses in a TCR-MHC interaction. Likewise, cell engagers (such as a bispecific antibody that e.g. binds CD3 on an effector cell with one arm and with the other arm an antigen on a target cell) which engage an effector cell with a target cell, can trigger the formation of a similar synapse structure as observed with a classic TCR-MHC interaction.

With regard to CARs of e.g. CAR T cells, these are chimeric antigen receptors that are to mimic a TCR or the like. CARs are engineered. The first generation of CAR were provided with an antigen recognition part often an antibody derived region (e.g. a scFv) fused to a transmembrane region and intracellular region of e.g. a CD3 ζ-chain. Later generations combined intracellular signalling domains from various costimulatory protein receptors (e.g., CD28, 41BB, ICOS) incorporated in the cytoplasmic tail of the CAR to enhance signalling further. Further generations also incorporated in their design an inducible release of transgenic immune modifiers, such as IL-12, to shape the tumour environment by augmenting e.g. T cell activation, attracting and activating innate immunity. As CARs often have antibody variable regions incorporated, these can target e.g. receptors themselves that are presented at the surface of a cell (e.g. Her2, PD-1 etc.), or can also target antigens presented by MHC, derived e.g. from proteins intracellular processed by the ubiquitin-proteasome system. Such peptides presented by MHC include proteins that are processed internally and presented by MHC, which can be derived from receptors, secreted proteins, intracellular proteins or internalized proteins. Synapses formed between CARs and target cells may not provide a classical bull's-eye like structure with a well-characterized SMAC domain, but may result in less organized pattern. Multiple CAR micro-clusters form and signalling molecules, which are dispersed in the centre of the synapse interface.

Synapse formation is a spatiotemporal process that starts e.g. by TCR binding to MHC or binding of an antigen with CAR or cell engagement, and subsequent phosphorylation of the cytosolic tails of CD3 resulting in a triggered state. This sets of a cascade of processes that result in an activated T cell state, which also depends on the effector cell type and its phenotypic state. Key processes include: calcium signalling, internal cell structure and/or cytoskeleton changes of effector cells, involving F-Actin, Talin, and changes in microtubules, centrosomes, lytic granules, nucleus position, and mitochondrial location. Transactivation of adhesion molecules, cytokine and marker expression. IFNγ, granzyme and perforin may be released by effector cells to thereby induce i.a. target cell killing. Also, in addition, in target cells apoptotic markers can be found, including ICAM-1 clustering, phosphatidyl translocation, mitochondrial depolarization, caspase-3 activation and DNA fragmentation. Hence, various stages of specific effector cell and target cell interaction and immune activation can be detected.

In any case, synapse formation, or a synapse can be determined by staining, i.e. with fluorescent labels, ligands, antibodies, or probes, or the like, which may be used on live cells or on fixed cells targeting the mechanisms involved in T cell activation and/or synapse formation. Sequencing based methods may also be used to identify activated T cells and thereby associate mRNA levels with the formation of stable synapses. T cell activation leads to changes in mRNA stability and expression. E.g. increases in expression of cytokine or secretory transcripts (IL2, IFNγ, granzyme, perforin) and proliferation pathways and either bulk or single-cell RNA sequencing may be used to detect these changes and correlate these to the number or synapse formed.

In any case, in the cells obtained, which can be either in the form of target cells bound to effector cells or separated cells, e.g. with trypsin, the marker associated with synapse formation can be determined. Thus, in a further embodiment, the marker associated with synapse formation is determined in either the target cell or the effector cell. In another embodiment, in the method in accordance with the invention one or more markers associated with synapse formation are determined and the one or more markers are determined in the effector cells and/or in the target cells. When cells are labelled, either in a singlet state or doublet state, cells may be highly advantageously be sorted and collected and subsequently analysed.

In one embodiment, a method in accordance with the invention is provided, wherein the marker associated with synapse formation is selected from the group consisting of calcium signalling signatures; spatial clustering of synapse localized molecules such as LFA-1, CD28, CD3, Agrin; changes to internal cell structure and/or cytoskeleton such as F-Actin, Talin, microtubules, centrosome, lytic granules, nucleus position, mitochondrial relocation; changes in effector cell motility; changes in external cell morphology and/or cell shape; and apoptosis of target cells. Synapse formation includes the initiation of a synapse up to and including the establishment of a synapse in which, as described above but not necessarily limited thereto, markers associated with synapse formation are associated.

In a further embodiment, the marker for synapse formation is calcium signalling, which can be detected with fluorescent calcium indicators, such as Fura2 AM (available from Invitrogen, item nr. F1221), which marker is suitable for detection in effector cells and in other live cells. Other suitable dyes to detect calcium signalling can be selected from the group of Fura Red AM, Indo-1, pentapotassium, Fluo-3, fluo-4, Calcium Green-1, Rhod-2 and X-Rhod-1, Oregon Green 488 BAPTA. Hence, in one embodiment, the marker for synapse formation is calcium signalling which is detected with an indicator selected from the group consisting of Fura2 AM, Fura Red AM, Indo-1, penta potassium, Fluo-3, fluo-4, Calcium Green-1, Rhod-2 and X-Rhod-1, and Oregon Green 488 BAPTA. With these markers calcium signalling can be detected which is a hallmark of synapse formation.

Furthermore, membrane potential dyes may also be used a calcium signalling indicators, such as the Invitrogen FluoVolt™ Membrane Potential Kit (catalog number: F10488). Depolarization of the synapse forming cells by using a slow-response potential-sensitive probe such as Invitrogen DiSBAC2(3) (Bis-(1,3-Diethylthiobarbituric Acid)Trimethine Oxonol), (catalog number: B413). Hence, in another embodiment the marker for synapse formation is detected utilizing membrane potential dyes.

In another embodiment, the marker for synapse formation is cytoskeleton rearrangement, which can be detected in effector cells with live or fixed cells, using cell staining, e.g. F-actin can be detected with Phalloidin conjugates or CellMask™ (Invitrogen, item nr. A57243). Other usable stains can include SiR-Actin, CellLight™ Talin-GFP, BacMam 2.0, or Tubulin Tracker Deep Red. Hence, in one embodiment, the marker for synapse formation is cytoskeleton rearrangement, which is detected with a stain selected from the group consisting of Phalloidin conjugates, CellMask™, SiR-Actin, Cell Light™ Talin-GFP, BacMam 2.0, or Tubulin Tracker Deep Red. With these markers, cytoskeleton rearrangement can be detected.

In another embodiment, the marker for synapse formation involves monitoring effector cell motility. Detection of effector cell motility can be performed by video/timelapse monitoring of effector cells that remain in contact with the target cells after the force has been exerted. Detection of synapse formation includes detecting effector cell immobility, i.e. upon synapse formation effector cells will stop moving and remain into contact with the target cell with which it forms a synapse. Cell motility can be detected by membrane staining of effector cells and imaging, or using brightfield, darkfield or phase contrast microscopy.

It is understood that for some of the methods for detecting a marker for synapse formation which use e.g. microscopy and monitoring of motility, or short-lived signals, may be combined with having cells provided with e.g. photoactivatable label, to, upon detection of the marker, activate such a label in the cell, such that in subsequent steps, e.g. sorting, FACS analysis or the like, cell for which the marker associated with synapse formation was detected can be easily be tracked.

As described above, in accordance with the invention, markers associated with synapse formation can be determined, e.g. via utilizing labels or the like. However, it may be highly advantageous to sequence obtained cells and identify synapse formation by sequencing. It is understood that sequencing comprises nucleotide sequencing, e.g. sequencing of DNA and/or RNA as expressed present in cells. Means and methods are widely known in the art to sequence DNA and/or RNA as expressed in the cell. Hence in another embodiment, in the methods in accordance with the invention the markers are determined with sequencing. This is in particular highly useful for such markers which are up- or downregulated in target cells and/or effector cells in forming a synapse or having a synapse. Suitable markers for T cell activation and synapse formation are transcripts linked to interferon expression, proliferation, and cytokine expression and include: Interferon pathway upregulation: CD4, IFIT3, IFIT2, STAT1, MX1, IRF7, ISG15, IFITM3, OAS2, JAK2, SOCS1, TRIM21; proliferation: LIF, IL2, CENPV, NME1, FABP5, ORC6, GOS2, GCK; cytokine expression: CCL3, IFNG, CCL4, XCL1, XCL2, CSF2, IL10, HOPX, TIM3, LAG3, PRF1, TNFRSF9, NKG7, IL26. Sequencing may also be used to identify non-synapse forming cells by detecting molecular signatures linked to resting cell states: FOXP3, CTLA4, MTNFRSF4, IRF4, BATF, TNFRSF18, TOX2, PRDMI, LEF1, ATM, SELL, KLF2, ITGA6, IL7R, CD52, S100A4, TGFB3, AQP3, NLRP3, KLF2, ITGB7.

It may be also advantageous to determine the molecular state of the target cells as this can give an indication of the cell killing potency of the effector cells. In the case of paired analysis where effector-target cell doublets are recovered, this enables direct linking of effector cell phenotype with their killing capabilities. Next to staining methods for apoptosis commonly used in the field, sequencing or qPCR can be used to detect transcripts linked to apoptosis or cell survival in the target cell, markers include: Bcl-2 family (BCL-xL) caspases 3, caspases 7, cleaved PARP, bax, bad, bak, bid, puma noxa, bcl-2, bcl-xl, mcl-1, p53, and cytochrome c. Smac/Diablo, survivn, Mcl-1, RNA Y1.

As it is understood that changes in gene expression may take some time and are not necessarily immediately detectable, one may allow after the step of exerting the force, the cells to remain bound to the target cells for some time and have these remain attached to the surface as well. Alternatively, one may also separate e.g. doublets that remained after exerting the force and subsequently e.g. sort doublets in single wells, and allow these to remain for some time. A suitable time to allow for expression of markers associated with synapse formation may be from 1 hour to 24 hours.

This is highly advantageous as sequencing methods allow for single cell sequencing, which also allows for determining the sequences of receptors expressed by the cell as well. Hence, in a further embodiment, different receptors are determined and identified via sequencing. In yet another embodiment, sequencing comprises single cell sequencing. In still another embodiment, sequencing comprises sequencing the expressed genome. Sequencing the expressed genome is highly useful as it allows to determine up- and downregulated gene expression. Nevertheless, sequencing genomic DNA may be useful as it may also provide useful information e.g. epigenetic markers associated with in vivo potency and durable response.

With regard to the cellular avidity score as determined herein in accordance with the invention, it is understood that this takes into account the number of cells that have remained bound to each other after applying the force and which may also take into account one or more markers associated with synapse formation. The cellular avidity score in accordance with the invention which takes into account target cells and effector cells associated with a marker may provide for a more accurate cellular avidity score which is more reflective of the function of effector cells, i.e. forming synapses with target cells. Hence, the cellular avidity score as determined herein in accordance with the invention may also be referred to as a functional cellular avidity score or a synaptic cellular avidity score. Alternatively, as the result of forming a synapse between an effector cell and a target cell is cell killing, the cellular avidity assay in accordance with the invention may also be an alternative to a potency assay. By measuring markers associated with synapse formation, the most potent, i.e. most strong, cell-cell interaction is determined. Hence, the cellular avidity as determined herein may also be referred to as a cellular avidity potency, and the cellular avidity score referred to as cellular avidity potency score.

As said, the target cells preferably are cancer cells or cells presenting an antigen. Other cells expressing an antigen, e.g. viral antigens or the like, may also be contemplated. An antigen can be presented by MHC, or the like. An antigen can also be presented at the cell surface e.g. as a (trans) membrane protein such as a receptor instead.

With regard to effector cells, in accordance with the invention, these are to carry a receptor, such as a T cell receptor (TCR) or a Chimeric Antigen Receptor (CAR). A T cell receptor (TCR) is a protein complex found on the surface of T cells, or T lymphocytes, that is responsible for recognizing an antigen, e.g. fragments of antigen as peptides bound to major histocompatibility complex (MHC) molecules. The TCR is composed of two different protein chains (it is a heterodimer). In humans, T cells mainly consists of an alpha (α) chain and a beta (β) chain. With regard to CARs of e.g. CAR T cells, these are chimeric antigen receptors that are to mimic a TCR or the like. CARs are engineered. The first generation of CAR were provided with an antigen recognition part often an antibody derived region (e.g. a scFv) fused to a transmembrane region and intracellular region of e.g. a CD3 ζ-chain. Later generations combined intracellular signalling domains from various costimulatory protein receptors (e.g., CD28, 41 BB, ICOS) incorporated in the cytoplasmic tail of the CAR to enhance signalling further. Further generations also incorporated in their design an inducible release of transgenic immune modifiers, such as IL-12, to shape the tumor environment by augmenting e.g. T cell activation, attracting and activating innate immunity. As CARs often have antibody variable regions incorporated, these can target e.g. receptors themselves that are presented at the surface of a cell (e.g. Her2, PD-1 etc.), or can also target antigens presented by MHC, derived e.g. from proteins intracellular processed by the ubiquitin-proteasome system. Such peptides presented by MHC include proteins that are processed internally and presented by MHC, which can be derived from receptors, secreted proteins, intracellular proteins or internalized proteins.

Effector cells, or immune effector cells, that may be contemplated in accordance with the invention may be selected from T lymphocytes, NK cells, monocytes, neutrophils, macrophages and dendritic cells. As shown in the example section, immune effector cells that may be contemplated in accordance with the invention include peripheral blood mononuclear cells (PBMCs), in particular the T cell population derived therefrom (CD3+), which comprises a mixture of helper (CD4+) or killer (CD8+) T cells. In a further embodiment, CD3+/CD8+ or CD3+CD4+ populations may be selected from PBMCs as immune effector cells. Such cells may be genetically modified, e.g. provided with e.g. a CAR, or may be primary cells, or derived therefrom, obtained from a subject, e.g. a human subject.

In another embodiment, a cell engager may be provided. Accordingly, in methods of the invention, a cell engager is provided capable of binding the effector cell and the target cell and inducing synapse formation, and steps of the methods include the cell engager, e.g. at least in the contacting step. Cell engagers include antibodies, or the like, which are capable of binding to a target cell and an effector cell. Such antibodies may include single chain antibodies comprising two binding domains such as scFv domain, and include BiTEs (i.e. bispecific T cell engagers) or the like. A conventional antibody design includes heavy and light chains, with one half of the antibody (one heavy chain and one light chain) engaging with a target cell, and the other half of the antibody (another heavy chain and another light chain) engaging with an effector cell, wherein preferably, the Fc domain is made inert. In any case, suitable cell engagers are widely known in the art and the current invention allows to study and/or determine cellular avidity, e.g. synapse formation, induced by a cell engager between a target cell and an effector cell. Hence, accordingly, in the means and methods in accordance with the invention, a cell engager may be provided in addition, and e.g. the cellular avidity score is determined, induced by said cell engager between an effector cell and a target cell, said cell engager having a binding region capable of binding the effector cell and a binding region capable of binding the target cell. It is understood that the contacting step in the methods of the invention can thus be performed in the presence of the cell engager to allow the cells to interact, i.e. effector cells and target cells, and form a bond, e.g. a synapse, via the cell engager.

In another embodiment, a method is provided comprising the steps in accordance with the method of the invention as herein defined, wherein the method is for use in screening and/or sorting effector cells. As shown in the example section, the means and the methods, and steps thereof, are suitable for sorting cells. Moreover, methods are provided for screening, e.g. comparing different effector cells and/or different receptors or different cell engager, as well. In such methods, different cell engagers or different effector cells, or different receptors can be ranked based on determined cellular avidity scores. Highly preferably, the force that is exerted in these methods was selected with the aim to have the cell-cell bonds that are retained after exerting the force, to consist substantially of specific cell-cell bonds in which a synapse was formed.

Of course, in another embodiment, in scenarios where it would be of interest to avoid aspecific binding, and determine these properties as well the means and methods may also be utilized to determine aspecific binding properties for particular interactions between effector cells and defined target cells. Hence, for example, the number of cells that did not bind, the number of cells that resulted in aspecific cell-cell bonds, and the number of cells that resulted in cell-cell bonds associated with a synapse may be determined as well. Such methods may also be performed with target cells to which the effector cells provided are not necessarily to have a specific interaction with, as the purpose of such further methods may involve e.g. to assess the risk of aspecific interactions e.g. of an effector cell.

In a further embodiment, the effector cells that are provided represent a heterogeneous cell population and wherein the method is for identifying from the heterogeneous cell population cell clones. Cell clones in the context of effector cells is understood to mean an effector cell, or cell clone, with a defined receptor sequence. It is thus understood that a cell clone with a receptor in accordance with the invention means a cell which expresses a defined receptor sequence, e.g. a defined CAR sequence or defined alpha and beta chains of a TCR, or the like. When of cell clones a plurality is provided, this is understood to comprise a plurality of cell receptors, i.e. receptors that are different with regard to their sequence. Hence, phrased differently, a heterogeneous population of cells accordingly is to comprise a plurality of different receptors, wherein a receptor can be presented by a cell clone population of cells, i.e. multiple cells carrying the same receptor.

For example, a library of CAR-T cells may be prepared from a lentiviral vector library encoding 100 different CAR sequences. With this vector library, 100,000 cells may be transduced. In accordance with the invention, transduced cells carrying the same CAR sequence may be referred to as a cell clone population, which population size is on average 1000 cells, and the plurality of cell clones may be referred to as 100. Of course, the transduced cells may be grown or expanded to 1000,000 cells, which increases the average size of a population to 10,000, whereas the plurality of cell clones, or cells carrying a unique receptor (construct) remains 100. Likewise, the same applies to e.g. primary cell samples, in which e.g. clonal expansion may have occurred e.g. in case of T cells or the like.

Accordingly, the current invention allows to identify from heterogeneous cell populations cell clones, e.g. via sequencing. Advantageously, one can also determine and quantify receptor sequences (or at least the variable region thereof) of a cell clone present in fractions instead (e.g. by determining copy number, e.g. per cell), the unique sequences of receptors identifying cell clones being of prime interest. By sequencing of cell fractions obtained, e.g. representative of the heterogeneous cell population provided to interact with the target cells, and subsequently a fraction obtained representing effector cells that were substantially forming a synapse, sequences representative of a cell clone population can be quantified in fractions. This way, cellular avidity scores can be determined from heterogeneous cell populations, without the need to resort to isolating individual cells, or cloning of TCR receptors or the like, and determining cellular avidity scores for separate receptors or separate effector cells. The more cells detected in fraction(s), such as bound and/or unbound fractions (i.e. attached and/or detached), the more confidence can be obtained with regard to cellular avidity scores, and/or relative cellular avidity.

Moreover, it was also observed that cells having different expression levels of receptors due to e.g. copy numbers, can provide for different cellular avidities. Accordingly, this means that by exerting a force in accordance with the invention, based on this difference, cells may remain bound to each other and/or cell-cell bonds may break at different rates at defined forces. This means that depending on expression levels, fractions can be enriched for cells with defined expression levels and the steps of the methods in accordance with the invention thus allow for enrichment of cells with defined expression levels. By e.g. repeating the enrichment method, highly enriched cell fractions with advantageous properties can thus be obtained. This may for example be highly advantageous in a scenario wherein a cell fraction is to be prepared for administration to a subject, e.g. a patient. This way, e.g. cells having e.g. predominantly a single copy can be obtained, which may be advantageous when e.g. higher copy numbers are associated with reduced efficacy, exhaustion or side effects.

With regard to the cells having different expression levels of a receptor, it is understood that this means that the cells are to express the same amino acid sequence at its cell surface where it is to interact with the target cells. There are means and methods known in the art with which expression levels of proteins and thus receptors can be well controlled. Different copy numbers of the same expression cassette can also be used as a means to provide for different expression levels. Promoter sequences driving expression of the receptors may be varied, resulting in different expression levels. Alternatively, 5′-UTR, 3′-UTR, splice signals and/or codon usage at the RNA level may also affect RNA stability and translation and thus variation therein may allow for different expression levels. Furthermore, other transcription and/or translation factors may have an effect on expression levels. Different inducible promoters may be utilized to vary expression level as well. In any case, different constructs can be made providing for different expression levels of the same receptor sequence, or cells can be genetically modified to achieve different expression levels of the same receptor sequence.

Accordingly, a method is provided in accordance with the invention, wherein the method comprises the steps as defined herein, and includes a separation step, wherein effector cells are provided having different expression levels of a receptor, and wherein the method is for providing enriched cell populations of effector cells having relatively higher or lower expression levels of the receptor. In a further embodiment, said method is for providing enriched cell population of effector cells having a defined copy number of a receptor, more preferably a copy number of 1.

Further embodiments may include methods being performed in the presence of agent(s). For example, one or more agents capable of modulating the cellular avidity between an effector cell and a target cell may be included in the steps of contacting the heterogenous population of cells with the target cells and subsequently applying a force. Modulating is understood to either enhance or reduce cellular avidity. This way, for example, in case an agent is present that is known to modulate a desired interaction between an effector cell and a target cell, receptor/target interactions may be selected that are not affected by such agents, or, conversely, are aided by such agents. Hence, in another embodiment, an agent capable of modulating the interaction between effector cells and target cells is included at least in the contacting step, said agent being capable of, or screened for, modulating the interaction between effector cells and a target cell, and/or the interaction between effector cell, cell engager and target cell.

In a further embodiment, cellular markers, e.g. related to synapse formation, and/or receptors are determined with sequencing. As shown in the example section, labels, such as fluorescent markers may be used, and these may be advantageous as it allows for the cells to remain. However, the methods in accordance with the invention may also use sequencing as a method to identify and/or determine the amount of effector cells and/or target cells, which may be used for determining cellular avidity and/or which may be of use in screening.

As said, a sequence of a receptor, or at least the variable region(s) thereof and also quantifying the number of cells representing such a receptor, but also of a marker such as a synaptic marker, may be determined utilizing sequencing methods (e.g. by using nucleic acid sequence quantification methods). A multitude of means and methods suitable therefor are known in the art (Pai, J. A. & Satpathy, A. T. (2021) Nature Methods, 18(8), 881-892). Engineered receptors such as used in CAR T cells generally use a single chain antibody sequence, therefor not requiring to identify receptor chain sequence pairs as is the case for TCRα and TCRβ chains. For combinations of receptor pairs, such as an alpha and beta chains, combinatorial pairing of receptors may be performed, e.g. when originating from a heterogeneous population. By multiplexing and deconvolution strategies, after sequencing the pairing of receptor chains may be determined by statistical analysis of the co-occurrence of receptor pairs in sub-fractions, using methods known in the art. Moreover, single-cell sequencing for pairing of receptor chains may be performed as well. Suitable sequencing methods in accordance with the invention that may be highly useful include sequencing the expressed genome and/or single cell sequencing.

### EXAMPLES

### Example 1

A monolayer of target Nalm6 (CD19+) cells (obtained from ATCC, product nr. CRL-1567) was brought in contact with IL-2 stimulated primary human effector T cells purified from buffy coat, which were transduced with CAR-FMC63 anti-CD19 (Kramer, A M; (2017) Delineating the impact of binding-domain affinity and kinetic properties on Chimeric Antigen Receptor T cell function. Doctoral thesis, UCL (University College London)). After removal of free and unbound cells by washing and centrifugation, cells remaining bound were resuspended and optionally trypsinized and subjected to FACS analysis.

### Monolayer Seeding

Nalm6 (CD19+) target cells were used, which were seeded as a monolayer to the ceiling of a 400 μm tall channel slide (μ-slides obtained from Ibidi, Cat. No: 80176). First, cells were counted and resuspended in serum-free medium at a concentration of approximately 30×10⁶ cells/mL in order to acquire a confluence close to 100%. Next, 100 μL of the resuspended cells was pipetted into the inlet of the channel slide and introduced in the channel by tilting the slide for few seconds until it reached the outlet of the channel slide. The channel slide was then placed horizontally so that the 100 μL filled the entire volume of the channel. The channel slide was flipped immediately, and incubated upside down in a humidity, temperature, and CO₂ controlled incubator for 30 minutes. After incubation, the medium was exchanged by first applying 60 μL of serum-containing medium into the inlet and subsequently withdrawing 60 μL from the outlet. This process was repeated four times. The slide was placed back upside down in the controlled incubator and incubated for another 30 minutes. Next, the medium was exchanged with PBS containing 0.5-1 μM CellTrace Violet (ThermoFisher #C34557) and incubated for 15 minutes in the incubator. Finally, the staining solution was exchanged by first applying 60 μL of the serum-containing medium into the inlet and subsequently withdrawing 60 μL from the outlet. This process was repeated four times.

### Effector Cell Preparation

In preparation of subsequent steps, primary T cells transduced with the anti-CD19 CAR-FMC63 were cultured. Cells were counted and viability was tested according to standard protocol. Next, 3×10⁶ cells/mL were stained with PBS containing 1 μM CellTracker™ Green CMFDA (Thermo Fisher #C2925) and incubated for 15 minutes in the incubator. Finally, the primary T cells were pelleted resuspended in complete medium.

### Effector Cell Binding

The channel inlet and outlet were filled with plugs prior to centrifugation in order to keep the liquid from moving out of the channel during centrifugation. The channel slide containing the seeded Nalm6 target cells was placed upright into a custom adaptor for the centrifuge bucket and spun for two minutes at 1000×g. Centrifugation removes fractions of the cells that were not well attached to the glass. Next, the slide was removed from the centrifuge. First, 100 μL of the effector cell suspension was pipetted into the inlet after which 100 μL was withdrawn from the outlet in order to obtain a homogenous distribution of effector cells throughout the length of the channel. The slide containing the target and effector cells was held upside down (to allow the effector cells and Nalm6 cells on the ceiling to interact) and incubated for five minutes. After incubation, three locations of the channel were imaged (one in the centre and one close to the inlet/outlet) using a fluorescent microscope. Each location was imaged in the brightfield, Green, and Violet channel. The slide was placed back in the centrifuge using the custom adapter and spun for two minutes at 1000×g. Finally, the channel slide was removed from the centrifuge and imaged at the same locations and using the same channels. It was observed that about 50% of the cells remained bound to the target cell after centrifugation. In general, low background binding is observed with Nalm6 cells when applying the centrifuge procedure, which usually is in the range of 5-15%.

### Cell Collection and FACS Analysis

After effector cell binding and imaging, the medium was exchanged by first applying 60 μL of serum-containing medium into the inlet and subsequently withdrawing 60 μl from the outlet. This process was repeated four times. The aliquots of removed medium from the outlet were discarded. Next, two 5 mL syringes were used to flush the target cells and the effector cells which were still attached to ceiling of the channel slide out of the channel. In order to do so, the plunger of one empty syringe was removed to act as a reservoir. The complete syringe was filled with 3 mL of complete medium and attached to the inlet. The empty syringe with the removed plunge was attached to the outlet. The syringe containing the complete medium was emptied in one vigorous movement, ensuring that all the contents moved through the channel and into the empty syringe. The flush was then reversed by pulling 3 mL back on the plunger of the complete syringe. The contents of the complete syringe were aliquoted into a clean 15 mL tube. The described flush procedure was repeated once more using the same two syringes and the final contents were pooled in the 15 mL tube. The cells in the 15 mL tube were pelleted by centrifugation at 400×g for five minutes. The medium was aspirated/decanted and the pellet was resuspended in serum-free medium.

The cell suspension was aliquoted in two equal parts into two clean tubes and each of the aliquots were pelleted by centrifugation at 400×g for five minutes. One pelleted aliquot was resuspended in 500 μL of Trypsin-EDTA (Thermo Fisher #25300054), and the other in 500 μL PBS.

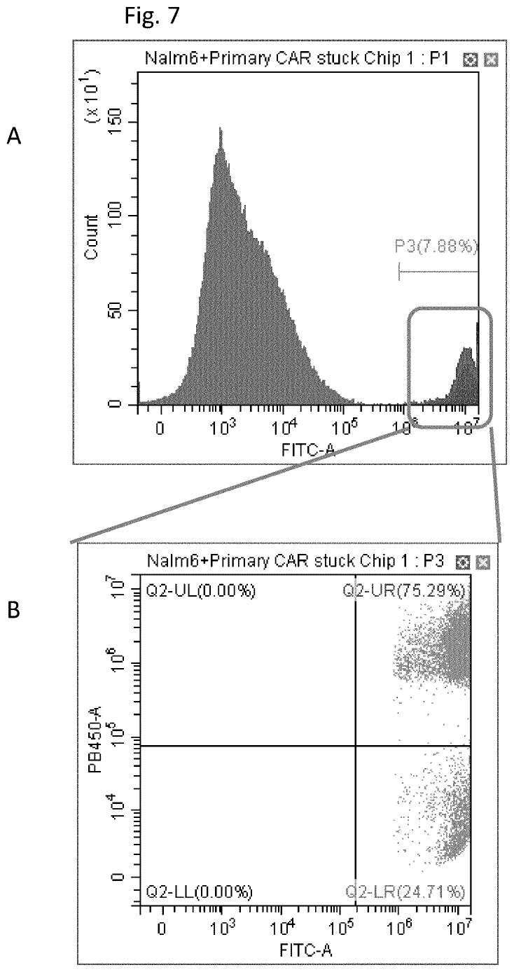

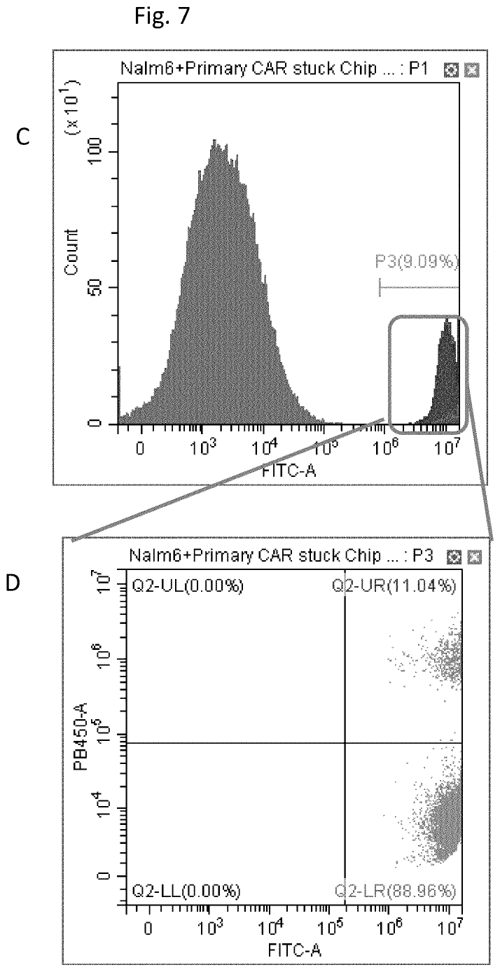

Both resuspended cell pellets were placed in the incubator for 10 minutes. Next, 5 mL of serum-containing medium was added to both tubes to dilute and inactivate the trypsin, if present, and the cells were pelleted by centrifugation at 400×g for five minutes. Both pellets were resuspended in FACS buffer (PBS, 0.5-1% BSA or 5-10% FBS, 0.1% NaN₃ sodium azide) and processed using a Flow Cytometer. CellTrace™ Violet was detected using a 405 nm laser and a 450/45 BF filter (PB450 channel), while CellTracker™ Green CMFDA was detected using a 488 nm laser and a 525/40 BF filter (FITC-A channel). See FIG. 7 showing the FACS plots thus obtained, further described below.

Utilizing the Flow Cytometer, cells of either PBS or Trypsin-EDTA treatment were first gated in order to remove debris. Around 100,000 events were gathered. No single cell gate was applied. Detection of the counts (i.e. CellTracker™ Green CMFDA) revealed a high intensity population in the FITC-A channel that was 7.88% and 9.09% of the total events that passed through the flow cytometer, respectively from the PBS control treated aliquot and the Trypsin treated aliquot (see FIGS. 7A and 7C and Table 1). The populations were assumed to comprise single T cells (“singlets”) and T cells bound to a Nalm6 cell (“doublets”). During the second gating, the subpopulation of the first gate was qualified based on the event having a signal in the FITC-A channel only, or in the FITC-A and PB450 channel simultaneously (see FIGS. 7B and 7D).

Of the PBS control treated aliquot, 75.29% of the counts were gated to the double positive channel (i.e PB450 and FITC-A), whereas 24.71% were gated to be positive to FITC-A only (see Table 1). Of the Trypsin treated aliquot, only 11.04% were gated to the double positive channel, whereas 88.96% was gated to be positive for FITC-A only (see Table 1). Counts derived from the trypsin treated aliquot presented as viable cells remain viable as judged from FACS analysis, including the observation that cells remained fluorescently labelled.

If we assume that for each aliquot, we started out with 1000 counts, this means that with PBS of the 1000 counts, 79 counts represented T cells, of which 59 T cells were bound to target cells, and 20 were T cells not bound to a target cell (see Table 2). With the Trypsin treated aliquot, 91 counts represented T cells of which 9 were bound, and 82 were not bound to a target cell (see Table 2).

It is remarked that the counts observed in the FITC-A channel are to originate from T cells that remained bound to target cells after centrifugation, and counts being negative for target cells (i.e. PB450-A) were to result from breaking the bond between T cells and target cells after the first centrifugation. From the results, it can be clearly observed that Trypsin-EDTA was able to efficiently cleave the bond between T cells and target cells, which is understood to comprise a highly substantial amount of synapses. Furthermore, because the PBS treated aliquot resulted in a substantial portion of the cells representing T cell singlets, this implies that the procedure of resuspension, and hence the force exerted, which is to exceed the force exerted during centrifugation (because the Nalm6 cells are detached from the channel slide in the procedure) caused further aspecific cell-cell bonds to break between T cells from target cells.

### Example 2

Nalm6 (CD19+) cells (obtained from ATCC, product nr. CRL-1567) were used as target cells. The Nalm6 (CD19+) cells were grown in serum-containing RPMI1664 medium, harvested and resuspended in serum-containing RPMI1664 medium.

Two effector cell populations were prepared. The first population contained untransduced Jurkat cells (obtained from ATCC, product nr. TIB-152; UNT) which served as a control. The second population contained CAR-transduced Jurkat cells.

The CAR-transduced Jurkat cells (CAR) were obtained by retroviral transduction of the untransduced Jurkat cells (obtained from ATCC, product nr. TIB-152) with a CAR-FMC63 anti-CD19 construct (see Kramer, AM; (2017) Delineating the impact of binding-domain affinity and kinetic properties on Chimeric Antigen Receptor T cell function. Doctoral thesis, UCL (University College London)). Transduction efficiency was about 85% as measured by median fluorescence intensity (MFI) using marker specific antibodies by means of methods generally known in the art. The effector cells were grown in serum-containing RPMI1664 medium, harvested and resuspended in serum-containing RPMI1664 medium.

In preparation of subsequent steps, the target cells and effector cells were counted and viability was tested according to standard protocols. Next, 1×10⁶ cells/population were stained with PBS containing 1 μM dye and incubated for 15 minutes in a humid incubator at 37° C. Finally, the cells were pelleted and resuspended in complete medium with all populations having the same concentration. The following dyes were used:
Target cells (Nalm6): CellTracker™ Green CMFDA (Thermo Fisher #C2925)
Effector cells (Jurkat UNT): CellTrace™ Violet (Thermo Fisher #C34557)
Effector cells (Jurkat CAR): CellTrace™ Far Red (Thermo Fisher #C34564)

Next, the following mixes were prepared (ratios were made based on volume as starting concentration of all populations was the same) and incubated for 30 minutes in a humid incubator at 37° C.:
1. Target cells (Nalm6):Effector cells (Jurkat UNT)
   10:1
2. Target cells (Nalm6):Effector cells (Jurkat CAR)
   10:1
3. Target cells (Nalm6):Effector cells (Jurkat UNT)
   1:1
4. Target cells (Nalm6):Effector cells (Jurkat CAR)
   1:1
5. Target cells (Nalm6):Effector cells (Jurkat UNT):Effector cells (Jurkat CAR)
   1:0.5:0.5

After incubation, the mixes were spun down at 400×G for 5 minutes and the obtained pellets were resuspended in PBS to obtain samples (each sample having the same concentration).

Thereafter, the samples were divided in half. The first half was left as is, while the second half was pipetted up and down 30 times using a 200 μl pipette. Each sample was read using a Flow Cytometer (100.000 cells were read per sample). CellTrace™ Violet was detected using a 405 nm laser and a 450/45 BF filter (PB450 channel), CellTracker™ Green CMFDA was detected using a 488 nm laser and a 525/40 BF filter (FITC channel) and CellTrace™ Far red was detected using a 638 nm laser and a 660/10 BF filter (APC channel).

During flow cytometry analysis, cells were first gated to remove debris (P1) and no single cell gate was applied. A histogram for the channel corresponding to an effector cell population, either Jurkat (UNT) (Violet, PB-450) or Jurkat (CAR) (Far Red, APC), was created and a positive effector cell subpopulation was selected (P2 and/or P3). The positive subpopulation contained single effector cells and effector cells still bound to target cells.

The positive subpopulation was then plotted to distinguish the fraction of events single positive for the effector cell channel (Q1-UL and Q2-UL) to the double positive in the effector cell and target cell channel (Q1-UR and Q2-UR). These events were created by the concomitant presence of at least one effector cell and one target cell.

The results are shown in Table 3. They demonstrate that, after pipetting, the fraction of double positive events decreased 12-36 times in the Jurkat UNT fraction, while only 1-2 times in the Jurkat CAR fraction. This shows that a force can successfully be applied such that aspecific cell-cell bonds (e.g. such as bonds between Jurkat UNT and Nalm6 target cells) are broken and synapses (e.g. such as bonds between Jurkat CAR and Nalm6 target cells) are retained.

TABLE 1

| | FITC-A (%) | PB450-A (+) (%) | PB450(−) (%) |
|---|---|---|---|
| PBS | 7.88 | 75.29 | 24.71 |
| Trypsin | 9.09 | 11.04 | 88.96 |

TABLE 2

| | FITC-A (%) | PB450-A (+) (%) | PB450(−) (%) |
|---|---|---|---|
| PBS | 79 | 59 | 20 |
| Trypsin | 91 | 9 | 82 |

TABLE 3

| | | Fraction of effector | Fraction of effector | |
|---|---|---|---|---|
| | | cells bound to | cells bound to | Reduction |
| | | target cells before | target cells after | of bound |
| | | pipetting (in %) | pipetting (in %) | cells |
| Mix 1 | | 10.8 | 0.7 | 15x |
| Mix 2 | | 62.1 | 53.7 | 1.15x |
| Mix 3 | | 15.0 | 0.4 | 36x |
| Mix 4 | | 36.2 | 17.8 | 2x |
| Mix 5 | UNT | 6.7 | 0.6 | 12x |
| | CAR | 50.3 | 23.9 | 2x |

1. A method for providing effector cells bound with target cells via a synapse comprising the steps of:
providing target cells;
providing effector cells;
contacting the target cells with the effector cells and allowing the cells to interact and form a synapse;
applying a force such that aspecific cell-cell bonds are broken and synapses are retained; thereby providing cells substantially comprised of target cells, effector cells, and effector cells bound with target cells via a synapse;
optionally, separating the effector cells bound with target cells via a synapse from the target cells and from the effector cells.

2. A method for providing effector cells bound with target cells via a synapse comprising the steps of:
providing target cells;
providing effector cells;
contacting the target cells with the effector cells and allowing the cells to interact and form a synapse;
applying a differential force in the range of 50 pN-10 nN that does not require attachment of target cells or effector cells;
thereby providing cells substantially comprised of target cells, effector cells, and effector cells bound with target cells via a synapse; and
optionally, separating the effector cells bound with target cells via a synapse from the target cells and from the effector cells.

3. A method for determining cellular avidity, comprising the further steps of:
determining the number of effector cells bound with target cells via a synapse, and, optionally, the number of effector cells and/or target cells; and
determine a cellular avidity score based on the number of effector cells bound with target cells via a synapse.

4. Method in accordance with claim 1, wherein the force applied is a shear force.

5. Method in accordance with claim 1, wherein the force applied is an ultrasonic force.

6. Method in accordance with claim 1, wherein the target cells or the effector cells are attached to a surface during the contacting step.

7. Method in accordance with claim 6, wherein after the contacting step, the cells that are not attached to the surface or not bound to the cells attached to the surface, are removed prior to applying the force.

8. Method in accordance with claim 1, wherein in the step of applying a force, an incremental force is applied and, optionally, fractions from different forces are obtained, and wherein the maximum force applied is such that aspecific cell bonds are broken and synapses are retained.

9. Method in accordance with claim 1, wherein effector cells bound with target cells via a synapse are separated from each other, preferably with trypsin.

10. Method in accordance with claim 1, wherein the cellular avidity score is determined by calculating the ratio of the number of effector cells associated with synapse formation to the number of effector cells that have interacted with the target cells in the contacting step; or by calculating the ratio of the number of effector cells associated with synapse formation to the number of effector cells bound to the target cells after the interaction step.

11. Method in accordance with claim 1, wherein in effector cells bound with target cells after the step of applying the force the presence of a marker associated with synapse formation is determined.

12. Method in accordance with claim 1, wherein the effector cells carry a receptor, such as a TCR or a CAR.

13. Method in accordance with claim 12, wherein the effector cells are selected from T lymphocytes, NK cells, monocytes, macrophages and dendritic cells.

14. Method in accordance with claim 1, wherein a marker and/or receptor is determined with sequencing.

15. Method in accordance with claim 14, wherein sequencing comprises sequencing the expressed genome and/or single cell sequencing.
